(19) United States
(12) Patent Application Publication
(10) Pub. No.: US 2025/0268651 A1
(43) Pub. Date: Aug. 28, 2025

(54) MEDICAL SYSTEMS AND METHODS FOR FAULT-TOLERANT TISSUE ABLATION

(71) Applicant: KARDIUM INC., Burnaby (CA)

(72) Inventors: Douglas Wayne GOERTZEN, New Westminster (CA); Daniel Martin REINDERS, Surrey (CA); Shane Fredrick MILLER-TAIT, North Vancouver (CA)

(21) Appl. No.: 19/058,354

(22) Filed: Feb. 20, 2025

Related U.S. Application Data
(60) Provisional application No. 63/556,579, filed on Feb. 22, 2024.

(51) Int. Cl. A61B 18/14; A61B 17/00; A61B 18/00; A61B 18/12

(52) U.S. Cl. CPC A61B 18/1492; A61B 2017/00154; A61B 2018/0016; A61B 2018/00577; A61B 2018/00702; A61B 2018/00767; A61B 2018/124; A61B 2018/1467; A61B 2560/0276

(57) ABSTRACT

A medical system may include a data processing device configured by a program, stored by a memory device system, at least to cause, via an input-output device system, a first group of electrodes to concurrently and collectively deliver as a group first energy configured to cause pulsed field ablation of bodily tissue; identify, at least in response to the first group of electrodes concurrently and collectively attempting to deliver as a group the first energy, that a fault condition has occurred; and cause, via the input-output device system and in response to identifying that the fault condition has occurred, the first group of electrodes to non-concurrently deliver, in separate subsets of electrodes, respective second energies to cause pulsed field ablation of bodily tissue, the separate subsets of electrodes including electrodes that collectively make up the first group of electrodes.

### CROSS-REFERENCE TO RELATED APPLICATION

This application claims the benefit of U.S. Provisional Application No. 63/556,579, filed Feb. 22, 2024, the entire disclosure of which is hereby incorporated herein by reference.

### TECHNICAL FIELD

Aspects of this disclosure generally are related to medical systems and methods for delivering tissue-ablative energy in a fault-tolerant manner.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with various methods including a technique known as the “PV (pulmonary vein) isolation”. Research has shown that atrial fibrillation typically begins in the pulmonary veins or at the point where they attach to the left atrium. There are typically four major pulmonary veins, and some or all may be a focal point for activity that may cause atrial fibrillation. During this procedure, physicians create specific patterns of lesions in the heart to block various paths taken by the spurious electrical signals. The patterns of lesions may include a pattern of one or more lesions that encircle at least one of the pulmonary veins. Lesions were originally created using incisions, but are now typically created by ablating the tissue with various techniques including radiofrequency (“RF”) ablation, microwave ablation, laser ablation, and cryogenic ablation.

Recently, a new ablation modality known as pulsed field ablation (PFA) has gained significant popularity in the ablation of various tissue structures, for example, in cardiac ablation. PFA is an ablation method that employs high voltage pulse delivery in proximity to target tissue. The electric field applied by the high voltage pulses in PFA physiologically changes the tissue cells to which the energy is applied (e.g., puncturing or perforating the cell membrane to form various pores therein). If a relatively low field strength is established, the formed pores may close in time and cause the cells to maintain viability (e.g., a process sometimes referred to as reversible electroporation). If relatively greater field strength is established, then permanent, and sometimes larger, pores form in the tissue cells, the pores allowing loss of control of ion concentration gradients (both inward and outward) thereby resulting in cell death (e.g., in a process sometimes referred to as irreversible electroporation). In contrast to thermal ablation techniques such as RF ablation and cryogenic ablation, PFA ablation is considered to be “non-thermal” in nature since the resulting tissue cellular death or destruction is not primarily or substantially dependent on thermal processes.

One important aspect of tissue ablation procedures is the duration of the procedure itself. Longer procedures tend to expose the patient to greater risk, so shortening procedure duration is a desirable design goal. However, delivering high energy capable of ablating tissue must be done safely to, e.g., ensure that the energy in a proper amount is delivered properly and to the target tissue, while reducing or eliminating damage to non-target tissue. Accordingly, tissue ablation systems include safety mechanisms capable of detecting a fault condition in the energy delivery circuit, which could cause delivery of excessive energy or insufficient energy to be delivered to target tissue. Conventionally, in a case where such a fault condition is detected, energy delivery is terminated until corrective action can be taken and the fault condition can be confirmed to be eliminated, which necessarily extends procedure time.

Accordingly, the present inventors recognized that there is a need in the art for improved medical systems that are configured to safely and effectively deliver tissue-ablative energy while continuing to reduce overall procedure time.

### SUMMARY

At least the above-discussed need is addressed and technical solutions are achieved in the art by various embodiments of the present invention. In some embodiments, a medical system may include a data processing device system; an input-output device system communicatively connected to the data processing device system, the input-output device system communicatively connectable to a plurality of electrodes supported by a structure of a catheter; and a memory device system communicatively connected to the data processing device system and storing a program executable by the data processing device system. The data processing device system may be configured by the program at least to cause, via the input-output device system, a first group of electrodes from the plurality of electrodes to concurrently and collectively deliver as a group first energy configured to cause pulsed field ablation of bodily tissue. The data processing device system may be configured by the program at least to identify, at least in response to the first group of electrodes concurrently and collectively attempting to deliver as a group the first energy to cause pulsed field ablation of bodily tissue, that a fault condition has occurred. The data processing device system may be configured by the program at least to cause, via the input-output device system and in response to identifying that the fault condition has occurred, the first group of electrodes to non-concurrently deliver, in separate subsets of electrodes, respective second energies to cause pulsed field ablation of bodily tissue, the separate subsets of electrodes comprising electrodes that collectively make up the first group of electrodes.

According to some embodiments, the separate subsets of electrodes may be separate pairs of electrodes. According to some embodiments, each of the separate subsets of electrodes may be a subset of two or more electrodes. According to some embodiments, each of the separate subsets of electrodes may be a subset of an even number of electrodes. According to some embodiments, each of the separate subsets of electrodes may include at least one electrode that is other than another electrode in another of the separate subsets of electrodes. According to some embodiments, each of the separate subsets of electrodes does not include any electrode that is also included in any of the other separate subsets of electrodes.

According to some embodiments, the first group of electrodes may include four or more electrodes. According to some embodiments, the first group of electrodes may include eight or more electrodes. According to some embodiments, the first group of electrodes may have an even number of electrodes.

According to some embodiments, the data processing device system may be configured by the program at least to receive, via the input-output device system, a signal set; and execute the identifying that the fault condition has occurred based at least on an analysis of the received signal set. According to some embodiments, the data processing device system may be configured by the program at least to receive, via the input-output device system, the signal set from an electrode set. According to some embodiments, the data processing device system may be communicatively connected to the plurality of electrodes via the input-output device system, and the electrode set may be from the plurality of electrodes. According to some embodiments, the electrode set may be from the first group of electrodes.

According to some embodiments, the fault condition may be a dielectric breakdown. According to some embodiments, the fault condition may be an overcurrent condition or a low impedance condition. According to some embodiments, the fault condition may be a low current condition or a high impedance condition.

According to some embodiments, the input-output device system may be communicatively connected to an energy source device system. The energy source device system may include a single power delivery driver configured to transmit energy to the first group of electrodes. The causing the first group of electrodes from the plurality of electrodes to concurrently and collectively deliver as a group the first energy configured to cause pulsed field ablation of bodily tissue may include causing the single power delivery driver to transmit the first energy to the first group of electrodes. According to some embodiments, the causing the first group of electrodes to non-concurrently deliver, in the separate subsets of electrodes, the respective second energies to cause pulsed field ablation of bodily tissue may include causing the single power delivery driver to transmit the respective second energies to the separate subsets of electrodes.

According to some embodiments, the input-output device system may be communicatively connected to an energy source device system. The energy source device system may include a plurality of power delivery drivers configured to transmit energy to the plurality of electrodes. According to some embodiments, the causing the first group of electrodes from the plurality of electrodes to concurrently and collectively deliver as a group the first energy configured to cause pulsed field ablation of bodily tissue may include causing the plurality of power delivery drivers to transmit the first energy to the first group of electrodes. According to some embodiments, the causing the first group of electrodes to non-concurrently deliver, in the separate subsets of electrodes, the respective second energies to cause pulsed field ablation of bodily tissue may include causing a first power delivery driver of the plurality of power delivery drivers to transmit a respective one of the respective second energies to a first subset of the separate subsets of electrodes, and may include causing a second power delivery driver of the plurality of power delivery drivers to transmit a respective one of the respective second energies to a second subset of the separate subsets of electrodes. According to some embodiments, the causing the first group of electrodes from the plurality of electrodes to concurrently and collectively deliver as a group the first energy configured to cause pulsed field ablation of bodily tissue may include causing one power delivery driver of the plurality of power delivery drivers to transmit the first energy to the first group of electrodes. According to some embodiments, the causing the first group of electrodes to non-concurrently deliver, in the separate subsets of electrodes, the respective second energies to cause pulsed field ablation of bodily tissue may include causing the one power delivery driver of the plurality of power delivery drivers to transmit the respective second energies to the separate subsets of electrodes. According to some embodiments, the causing the first group of electrodes to non-concurrently deliver, in the separate subsets of electrodes, the respective second energies to cause pulsed field ablation of bodily tissue may include causing a first power delivery driver of the plurality of power delivery drivers to transmit a respective one of the respective second energies to a first subset of the separate subsets of electrodes, and may include causing a second power delivery driver of the plurality of power delivery drivers to transmit a respective one of the respective second energies to a second subset of the separate subsets of electrodes.

According to some embodiments, the data processing device system may be communicatively connected to the plurality of electrodes via the input-output device system.

According to some embodiments, the separate subsets of electrodes may include at least a first subset of electrodes and a second subset of electrodes, and the causing the first group of electrodes to non-concurrently deliver, in the separate subsets of electrodes, the respective second energies to cause pulsed field ablation of bodily tissue may include causing: (a) a delivery of a first portion of the respective second energy delivered by the second subset of electrodes to be delivered after a delivery of a first portion of the respective second energy delivered by the first subset of electrodes, and (b) a delivery of a second portion of the respective second energy delivered by the first subset of electrodes to be delivered after the delivery of the first portion of the respective second energy delivered by the second subset of electrodes. According to some embodiments, the first portion of the respective second energy delivered by the first subset of electrodes may be a first voltage pulse set, the first portion of the respective second energy delivered by the second subset of electrodes may be a second voltage pulse set, and the second portion of the respective second energy delivered by the first subset of electrodes may be a third voltage pulse set. According to some embodiments, (i) the first voltage pulse set, (ii) the second voltage pulse set, (iii) the third voltage pulse set, each of (i) and (ii), each of (i) and (iii), each of (ii) and (iii), or each of (i), (ii), and (iii) may be a pulse set of only a single voltage pulse. According to some embodiments, the single voltage pulse may be a single biphasic voltage pulse.

According to some embodiments, the respective second energies may be configured to be within 10% of a portion of the first energy that was not delivered due at least to the identified fault condition.

According to some embodiments, the first energy and each respective second energy may be configured as a train of voltage pulses.

According to some embodiments, the first energy may be configured as a train of voltage pulses.

According to some embodiments, each respective second energy may be configured as a sequence of voltage pulse sets, an inter-pulse delay between pulses in each voltage pulse set in the sequence of voltage pulse sets may be less than an inter-pulse-set delay between voltage pulse sets in the sequence of voltage pulse sets. According to some embodiments, the causing the first group of electrodes to non-concurrently deliver, in the separate subsets of electrodes, the respective second energies to cause pulsed field ablation of bodily tissue may include causing the separate subsets of electrodes to non-concurrently deliver the respective second energies at least by causing cycling among the voltage pulse sets of the sequences of voltage pulse sets of the respective second energies. According to some embodiments, the causing the first group of electrodes to non-concurrently deliver, in the separate subsets of electrodes, the respective second energies to cause pulsed field ablation of bodily tissue may include causing the separate subsets of electrodes to non-concurrently deliver the respective second energies at least by interleaving the voltage pulse sets of the sequences of voltage pulse sets of the respective second energies. According to some embodiments, each of at least one voltage pulse set of the voltage pulse sets of the sequences of voltage pulse sets of the respective second energies may be a pulse set of only a single voltage pulse. According to some embodiments, the single voltage pulse may be a single biphasic voltage pulse. According to some embodiments, each of the voltage pulse sets of the voltage pulse sets of the sequences of voltage pulse sets of the respective second energies may be a pulse set of only a single voltage pulse. According to some embodiments, the single voltage pulse may be a single biphasic voltage pulse.

According to some embodiments, the data processing device system may be configured by the program at least to identify that the fault condition has occurred in a particular subset of the separate subsets of electrodes at least in response to at least the particular subset of the separate subsets of electrodes attempting to deliver the respective second energy.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

For example, in some embodiments, a method may be executed by a data processing device system according to a program stored by a communicatively connected memory device system, the data processing device system also communicatively connected to an input-output device system, and the method may include causing, via the input-output device system, a first group of electrodes from the plurality of electrodes to concurrently and collectively deliver as a group first energy configured to cause pulsed field ablation of bodily tissue. The method may include identifying, at least in response to the first group of electrodes concurrently and collectively attempting to deliver as a group the first energy to cause pulsed field ablation of bodily tissue, that a fault condition has occurred. The method may include causing, via the input-output device system and in response to identifying that the fault condition has occurred, the first group of electrodes to non-concurrently deliver, in separate subsets of electrodes, respective second energies to cause pulsed field ablation of bodily tissue, the separate subsets of electrodes comprising electrodes that collectively make up the first group of electrodes.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

For example, in some embodiments, one or more computer-readable storage mediums may store a program executable by a data processing device system communicatively connected to an input-output device system. The program may include first delivery instructions configured to cause, via the input-output device system, a first group of electrodes from the plurality of electrodes to concurrently and collectively deliver as a group first energy configured to cause pulsed field ablation of bodily tissue. The program may include identification instructions configured to cause identification, at least in response to the first group of electrodes concurrently and collectively attempting to deliver as a group the first energy to cause pulsed field ablation of bodily tissue, that a fault condition has occurred. The program may include second delivery instructions configured to cause, via the input-output device system and in response to identifying that the fault condition has occurred, the first group of electrodes to non-concurrently deliver, in separate subsets of electrodes, respective second energies to cause pulsed field ablation of bodily tissue, the separate subsets of electrodes comprising electrodes that collectively make up the first group of electrodes.

In some embodiments, each of any of one or more or all of the computer-readable storage mediums or medium systems (also referred to as processor-accessible memory device systems) described herein is a non-transitory computer-readable (or processor-accessible) data storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 includes a schematic representation of a medical system according to various example embodiments, the medical system including a data processing device system, an input-output device system, and a memory device system.

FIG. 2 includes a cutaway diagram of a heart showing a transducer-based device percutaneously placed in a left atrium of the heart, according to various example embodiments.

FIG. 3A includes a partially schematic representation of a medical system according to various example embodiments, the medical system including a data processing device system, an input-output device system, a memory device system, and a transducer-based device including a plurality of transducers and an expandable structure shown in a delivery or unexpanded configuration.

FIG. 3B includes the representation of the medical system of FIG. 3A with the expandable structure shown in a deployed or expanded configuration, according to some embodiments.

FIG. 3C includes a portion of the medical system of FIG. 3B as viewed from a different viewing direction, according to some embodiments.

FIG. 4 includes a schematic representation of a transducer-based device that includes a flexible circuit structure, according to various example embodiments.

FIG. 5 includes a block diagram of various methods for fault-tolerant tissue ablation, according to some embodiments, such methods implemented by one or more of the various medical systems described herein, according to some embodiments.

FIGS. 6A, 6B, and 6C each respectively illustrate characteristics of a respective portion of a respective high voltage pulse train for pulsed field ablation, according to some embodiments.

FIGS. 7A and 7B illustrate an example of a first group of electrodes concurrently and collectively delivering first energy configured to cause pulsed field ablation, according to some embodiments.

FIGS. 8A and 8B illustrate an example of a first subset from the first group of electrodes delivering, in response to an identification that a fault condition has occurred in the delivery of the first energy by the first group of electrodes, a respective second energy configured to cause pulsed field ablation, according to some embodiments.

FIGS. 9A and 9B illustrate an example of a second subset from the first group of electrodes delivering, in response to the identification that the fault condition has occurred in the delivery of the first energy by the first group of electrodes, a respective second energy configured to cause pulsed field ablation, according to some embodiments.

FIG. 10 illustrates a cycling of deliveries of the respective second energies by respective subsets of electrodes from the first group of electrodes, according to some embodiments.

### DETAILED DESCRIPTION

At least the above-discussed need is addressed, and technical solutions are achieved by various embodiments of the present invention. In some embodiments, tissue-ablative energy is transmitted to an identified group of electrodes and, if a fault condition is detected in the transmission of that energy, then that group of electrodes is divided into subsets of electrodes and tissue-ablative energy is delivered to those subsets of electrodes separately. At least such an approach allows the fault condition to be isolated to a smaller number of electrodes as compared to the state where the tissue-ablative energy was delivered to the entire group of electrodes. Further, if the fault condition still exists during the transmission of the tissue-ablative energy to the separate subsets of electrodes and transmission of the tissue-ablative energy to the subset(s) of electrodes that are impacted by the fault condition must be terminated, then the tissue-ablative energy may still be transmitted to the subset(s) of electrodes that are not impacted by the fault condition so that the medical procedure can still proceed in some manner. Accordingly, it can be seen that at least some aspects of the present invention can advantageously allow fault condition detection and isolation to occur concurrently with the transmission of tissue-ablative energy to electrodes that are not impacted by the fault condition, so that tissue-ablative energy can be safely delivered while reducing overall procedure time at least in the case where a fault condition occurs.

It should be noted that various embodiments of the invention are not limited to these features and benefits, which are referred to for purposes of illustration only, and additional and alternative features and benefits will become apparent from the following description in conjunction with reference to the figures.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments. In one embodiment, all references to “some embodiments” may refer to the same single embodiment.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers or is a necessary precondition for the event A, according to various embodiments.

In some embodiments, the word “adjacent”, the word “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the word “adjacent”, the word “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In some embodiments, the word “adjacent”, the word “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the word “adjacent”, the word “proximate”, and the like are not limited to any of the above example definitions, according to some embodiments. In addition, the word “adjacent” and the word “proximate” do not have the same definition, according to some embodiments.

The phrase “pulsed field ablation” (“PFA”) as used in this disclosure refers, in some embodiments, to an ablation method that employs high voltage pulse delivery in a unipolar or bipolar ablation mode in proximity to target tissue. In some embodiments, each high voltage pulse may be referred to as a discrete energy application. In some embodiments, a grouped plurality of high voltages pulses (e.g., a train or packet of voltage pulses) may be referred to as a discrete energy application. Each high voltage pulse may be a monophasic pulse including a single polarity, or a biphasic pulse including a first component having a first particular polarity and a second component having a second particular polarity opposite the first particular polarity. Each of the first component and the second component of a biphasic pulse may be referred to as a monophasic pulse, such that a biphasic pulse may be considered to be made of two monophasic pulses of opposite polarity, in some embodiments. In some embodiments, the second component of the biphasic pulse follows immediately after the first component of the biphasic pulse. In some embodiments, the first and second components of the biphasic pulse are temporally separated by a relatively small time interval (e.g., an inter-phase delay). In some embodiments, successive monophasic or biphasic pulses are separated by a period of time referred to as an inter-pulse delay. In the case where a biphasic pulse is considered to be made of two monophasic pulses of opposite polarity, a delay between such two monophasic pulses would be considered an inter-phase delay, whereas a delay between the last of such two monophasic pulses and the first monophasic pulse of the next biphasic pulse would be considered an inter-pulse delay. In some embodiments, the duration of the inter-pulse delay may be greater than the duration of each of the monophasic or biphasic pulses. In some embodiments, each high voltage pulse may include a multiphasic pulse, such as a triphasic pulse, that includes a first component having a first particular polarity (e.g., which may be referred to as a first monophasic pulse of the first particular polarity), a second component having a second particular polarity (e.g., which may be referred to as a second monophasic pulse of the second particular polarity) opposite the first particular polarity, and a third component having a third particular polarity (e.g., which may be referred to as a third monophasic pulse of the third particular polarity) that is the same as the first particular polarity.

The word “proximal”, in the context of a proximal portion, proximal location, and the like of a medical device, includes, for example, the portion, location, and the like, being or being configured to be further away from a patient or portion of or region within a patient (e.g., a bodily cavity) intended to be treated or assessed by the medical device, as compared to a distal portion, location, and the like of the medical device, according to some embodiments. In some embodiments, the word “proximal”, in the context of a proximal portion, proximal location, and the like of a medical device, includes, for example, the portion, location, and the like, being or being configured to be delivered (e.g., percutaneously or intravascularly) toward a patient or portion of or region within a patient (e.g., a bodily cavity) intended to be treated or assessed by the medical device, after or behind a distal portion, location, and the like of the medical device. On the other hand, the word “distal”, in the context of a distal portion, distal location, and the like of a medical device, includes, for example, the portion, location, and the like, being or being configured to be closer to a patient or portion of or region within a patient (e.g., a bodily cavity) intended to be treated or assessed by the medical device, as compared to a proximal portion, location, and the like of the medical device, according to some embodiments. In some embodiments, the word “distal”, in the context of a distal portion, distal location, and the like of a medical device, includes, for example, the portion, location, and the like, being or being configured to be delivered (e.g., percutaneously or intravascularly) toward a patient or portion of or region within a patient (e.g., a bodily cavity) intended to be treated or assessed by the medical device, before or ahead of a proximal portion, location, and the like of the medical device.

According to some embodiments, the word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intra-cardiac cavities (e.g., a left atrium or right atrium).

According to some embodiments, the phrase “bodily opening” as used in this disclosure should be understood to include, for example, a naturally occurring bodily opening or channel or lumen; a bodily opening or channel or lumen or perforation formed by an instrument or tool using techniques that can include, but are not limited to, mechanical, thermal, electrical, chemical, and exposure or illumination techniques; a bodily opening or channel or lumen or perforation formed by trauma to a body; or various combinations of one or more of the above. Various elements having respective openings, lumens, or channels and positioned within the bodily opening (e.g., a catheter sheath or catheter introducer) may be present in various embodiments. These elements may provide a passageway through a bodily opening for various devices employed in various embodiments.

The phrase “bodily cavity” as used in this disclosure should be understood to mean a cavity in a body. The bodily cavity may be a cavity or chamber provided in a bodily organ (e.g., an intra-cardiac cavity or chamber of a heart). The bodily cavity may be provided by a bodily vessel.

The word “tissue” as used in this disclosure should be understood to include, for example, any surface-forming tissue that is used to form a surface of a body or a surface within a bodily cavity, a surface of an anatomical feature or a surface of a feature associated with a bodily opening positioned in fluid communication with the bodily cavity. The tissue may include, for example, part or all of a tissue wall or membrane that defines a surface of the bodily cavity. In this regard, the tissue may form an interior surface of the cavity that surrounds a fluid within the cavity. In the case of cardiac applications, tissue may include, for example, tissue used to form an interior surface of an intra-cardiac cavity such as a left atrium or right atrium. In some embodiments, tissue is non-excised tissue. In some embodiments, the word “tissue” may refer to a tissue having fluidic properties (e.g., blood) and may be referred to as fluidic tissue.

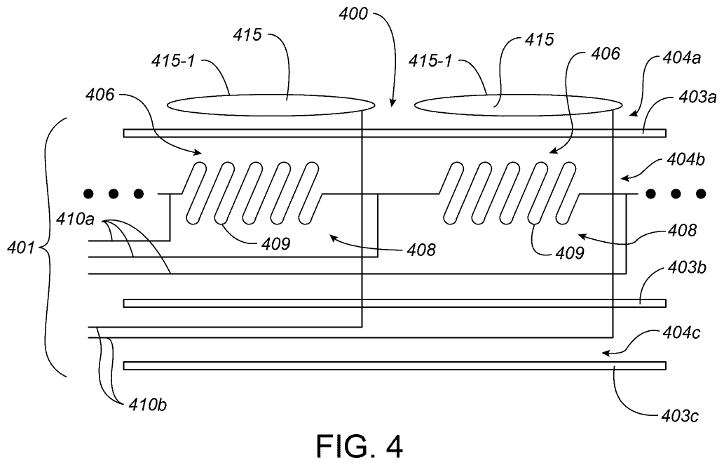

According to some embodiments, the word “transducer” as used in this disclosure should be interpreted broadly as any device configured to transmit or deliver energy; distinguish between fluid and tissue; sense temperature; generate heat; ablate tissue; sense, sample, or measure electrical activity of a tissue surface (e.g., sense, sample, or measure intracardiac electrograms, or sense, sample, or measure intracardiac voltage data); stimulate tissue; provide location information (e.g., in conjunction with a navigation system); or any combination thereof. A transducer may convert input energy of one form into output energy of another form. Without limitation, a transducer may include, for example, an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 4 discussed below.

The term “activation” as used in this disclosure, according to some embodiments, should be interpreted broadly as making active a particular function as related to various transducers such as those disclosed herein, for example. Particular functions can include, but are not limited to, tissue ablation; sensing, sampling, or measuring electrophysiological activity (e.g., sensing, sampling, or measuring intracardiac electrogram information, or sensing, sampling, or measuring intracardiac voltage data); sensing, sampling, or measuring temperature; and sensing, sampling, or measuring electrical characteristics (e.g., tissue impedance or tissue conductivity). For example, in some embodiments, activation of a tissue ablation function of a particular transducer or electrode is initiated by causing energy sufficient to cause tissue ablation to be delivered to the particular transducer or electrode from an energy source device system (also known as a power supply system in some embodiments). In some embodiments, activation of a tissue ablation function of a particular transducer or electrode is initiated by causing energy sufficient for tissue ablation to be delivered by the particular transducer or electrode. Alternatively, in some embodiments, the activation can be deemed to be initiated when the particular transducer or particular electrode causes tissue that is to be ablated to exhibit tissue-ablative damage. In some embodiments, the activation can last for a duration concluding when the ablation function is no longer active, such as when energy sufficient for the tissue ablation is no longer delivered or provided to, or transmitted by, the particular transducer or particular electrode. Alternatively, in some embodiments, the activation period can be deemed to be concluded when the tissue that is being ablated no longer accrues tissue-ablative damage, which may be due to a reduction or cessation of the energy provided or transmitted by the energy source device system or delivered by the particular transducer or electrode. In some contexts and embodiments, however, the word “activation” may merely refer to the initiation of the activating of a particular function, as opposed to referring to both the initiation of the activating of the particular function and the subsequent duration in which the particular function is active. In these contexts, the phrase or a phrase similar to “activation initiation” may be used. For example, in some embodiments, activation initiation may cause initiation of a delivery of energy (e.g., energy sufficient for tissue ablation) from a particular transducer or electrode.

Some embodiments of the present invention may be implemented at least in part by a data processing device system or a controller system configured by a software program. Such a program may equivalently be implemented as multiple programs, and some or all of such software program(s) may be equivalently constructed in hardware. Reference to “a program” should be interpreted to include one or more programs.

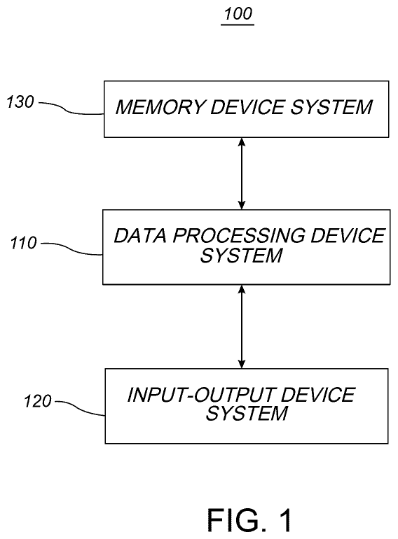

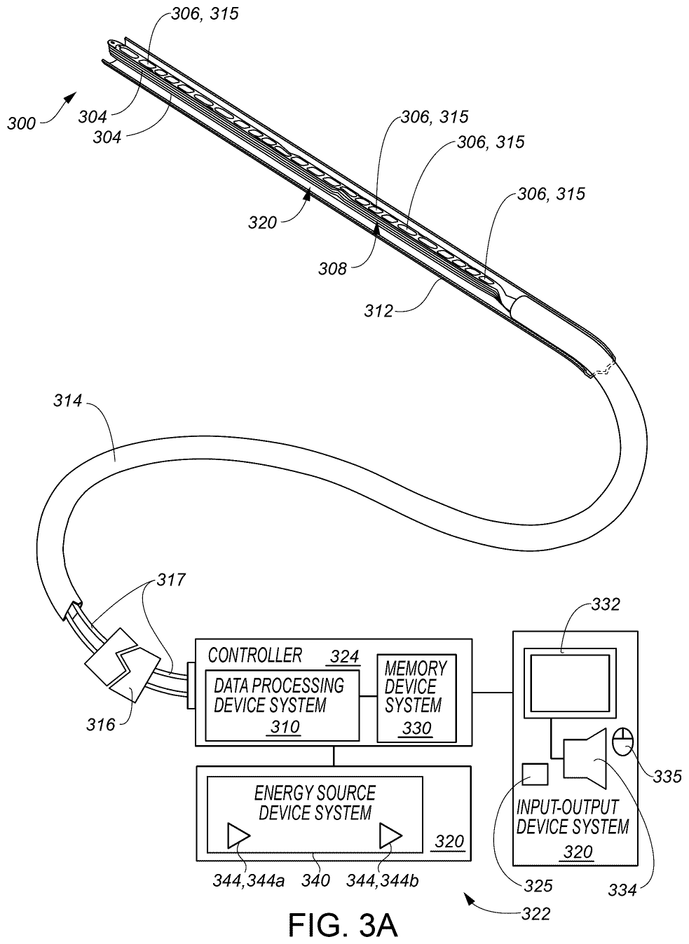

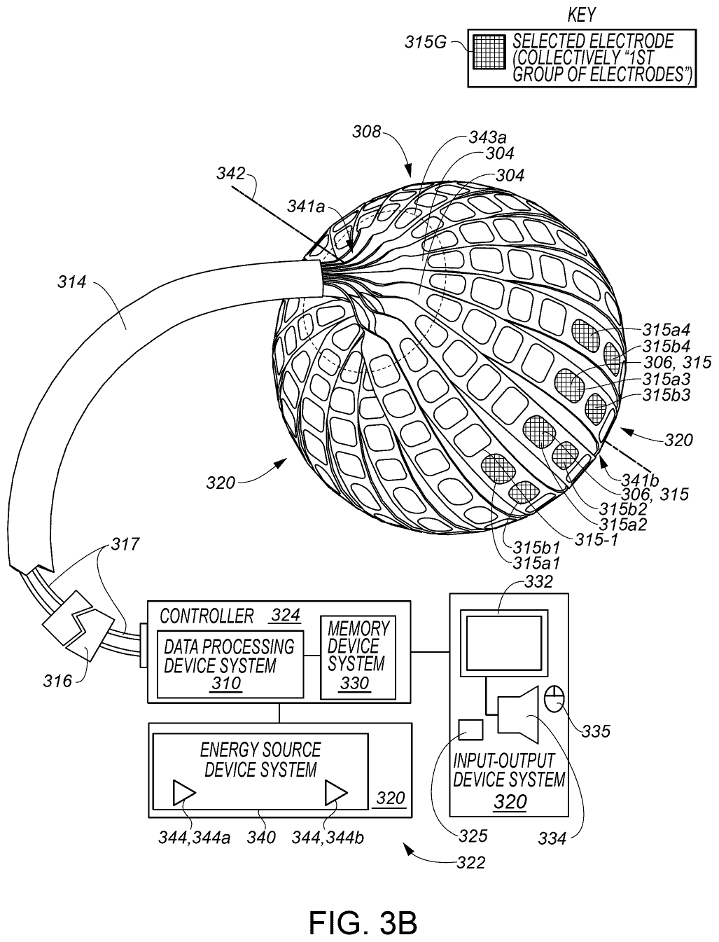

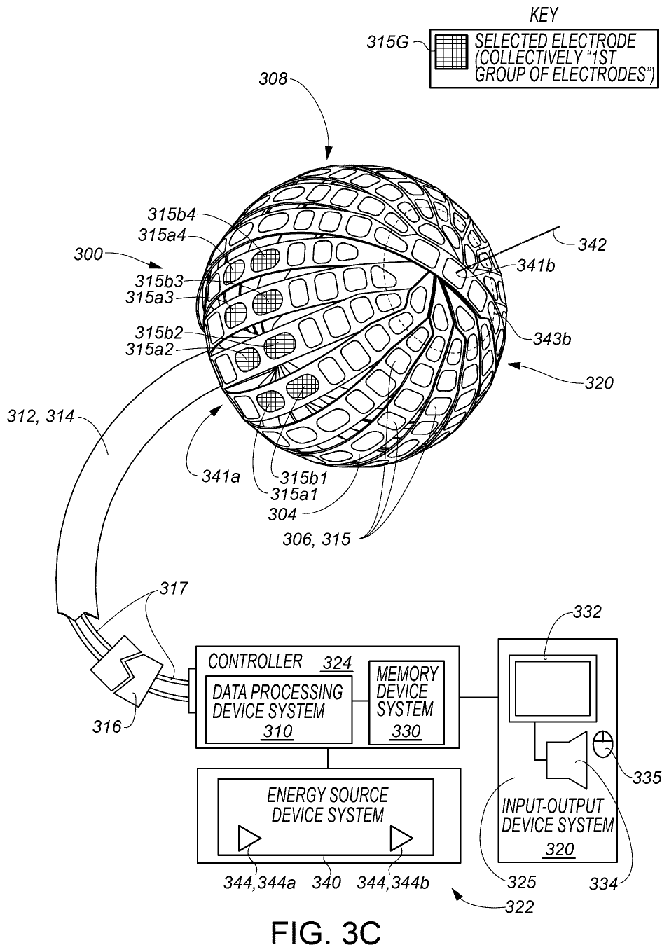

According to some embodiments, the term “program” in this disclosure should be interpreted to include one or more programs including a set of instructions or modules that may be executed by one or more components in a system, such as a controller system or data processing device system, in order to cause or configure the system to perform one or more operations. The set of instructions or modules may be stored by any kind of memory device, such as those described subsequently with respect to the memory device system 130, 330, or both, shown in FIGS. 1 and 3, respectively. In addition, this disclosure may describe or similarly describe that the instructions or modules of a program are configured to cause the performance of an action. The phrase “configured to” in this context is intended to include, for example, at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). In some instances, this disclosure may describe that the instructions or modules of a program perform an action. Such descriptions should be deemed to be equivalent to describing that the instructions or modules are configured to cause the performance of the action. The term “module” may be defined as a set of instructions. The term “program” and the term “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information, signals, or data or the like includes modifications to that information, signals, or data. For example, “data X” may be encrypted for transmission, and a reference to “data X” is intended to include both its encrypted and unencrypted forms, unless otherwise required or indicated by context. For another example, “image information Y” may undergo a noise filtering process, and a reference to “image information Y” is intended to include both the pre-processed form and the noise-filtered form, unless otherwise required or indicated by context. In other words, both the pre-processed form and the noise-filtered form are considered to be “image information Y”, unless otherwise required or indicated by context. In order to stress this point, the phrase “or a derivative thereof” or the like may be used herein. Continuing the preceding example, the phrase “image information Y or a derivative thereof” refers to both the pre-processed form and the noise-filtered form of “image information Y”, unless otherwise required or indicated by context, with the noise-filtered form potentially being considered a derivative of “image information Y”. However, non-usage of the phrase “or a derivative thereof” or the like nonetheless includes derivatives or modifications of information or data unless otherwise explicitly noted or required by context.

In some embodiments, the phrase “graphical representation” used herein is intended to include a visual representation presented via a display device system and may include computer-generated text, graphics, animations, or one or more combinations thereof, which may include one or more visual representations originally generated, at least in part, by an image-capture device, such as computerized tomography (“CT”) scan images, magnetic resonance imaging (“MRI”) images, or images created from a navigation system (e.g., an electro-potential navigation system or an electro-magnetic navigation system), according to some embodiments. The graphical representation may include various entities depicted in a three-dimensional manner, in some embodiments. The graphical representation may include various entities depicted in a two-dimensional manner that are mapped from a three-dimensional space into a two-dimensional coordinate system, in some embodiments.

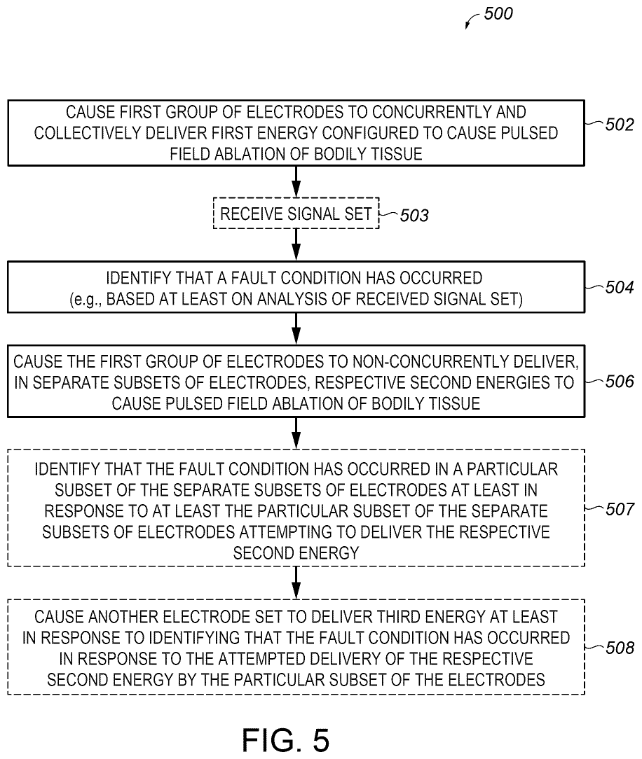

Example methods are described herein with respect to FIG. 5. Such figure is described to include blocks associated with actions, computer-executable instructions of one or more programs, or both actions and computer-executable instructions, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in FIG. 5 is not limited to an actual structure of any program or set of instructions or required ordering of method tasks, and such method figure, according to some embodiments, merely illustrates the tasks that instructions are configured to perform, for example, upon execution by a data processing device system in conjunction with interactions with one or more other devices or device systems.

FIG. 1 schematically illustrates a portion of a transducer-activation system or controller system thereof 100 that may be employed to at least select, control, activate, or monitor a function or activation of a number of electrodes or transducers (e.g., ablation transducers configured to cause thermal ablation or ablation transducers configured to cause PFA), according to some embodiments. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIG. 5. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, quantum components, or otherwise.

The memory device system 130 includes one or more processor-accessible memory devices configured to store one or more programs and information, including the program(s) and information needed to execute the methods or functions described herein, including those described with respect to FIG. 5. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, quantum, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” may include or may be a non-transitory processor-accessible (or computer-readable) data storage medium. In some embodiments, the processor-accessible memory device system 130 may include or may be a non-transitory processor-accessible (or computer-readable) data storage medium system. In some embodiments, the memory device system 130 may include or may be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In at least the case where the data processing device system 110, the input-output device system 120, and the memory device system 130 are located within the same device, the system 100 of FIG. 1 may be implemented by a single application-specific integrated circuit (ASIC), field programmable gate array (FPGA), system on chip (SOC), or other type of integrated circuit, in some embodiments. In this regard, in some embodiments, the processor-accessible memory device system 130 may be considered to be integrated with the data processing device system 110, such that circuitry or hardware may itself be encoded with the equivalent of computer-executable program instructions described herein to execute the methods and actions described herein, including those described with respect to the methods 500 of FIG. 5. Such integration may be considered a type of communicative connection between the data processing device system 110 and the processor-accessible memory device system 130. Such integration may also be considered control circuitry or a controller that combines at least some functionality of the processor-accessible memory device system 130 and the data processing device system 110, such that the control circuitry or controller is communicatively connected to the input-output device system 120 and is configured (e.g., via circuitry or hardware encoded with the equivalent of computer-executable program instructions) at least to perform the methods and actions described herein, including those described with respect to the methods 500 of FIG. 5.

The input-output device system 120 may include a mouse, a keyboard, a touch screen, another computer, a processor-accessible memory device system, a network-interface card or network-interface circuitry, or any device or combination of devices from which a desired selection, desired information, instructions, or any other data is input to the data processing device system 110. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device (e.g., a catheter, or portion thereof) that includes a spatial distribution of electrodes. The phrase “transducer-based device” or “transducer-based device system” is intended to include one or more physical systems that include various transducers (e.g., electrodes).

The input-output device system 120 also may include an image generating device system, a display device system, a speaker or audio output device system (e.g., speaker or audio output device system 334 shown in FIGS. 3A, 3B, and 3C, discussed below), a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In some embodiments, the input-output device system 120 may include a transducer-based device, as discussed above, and in some embodiments, the transducer-based device may act as a device or device system that provides information to, receives instructions or energy from, or both provides information to and receives instructions or energy from the data processing device system 110. In this regard, the input-output device system 120 may include various devices or systems described in various embodiments.

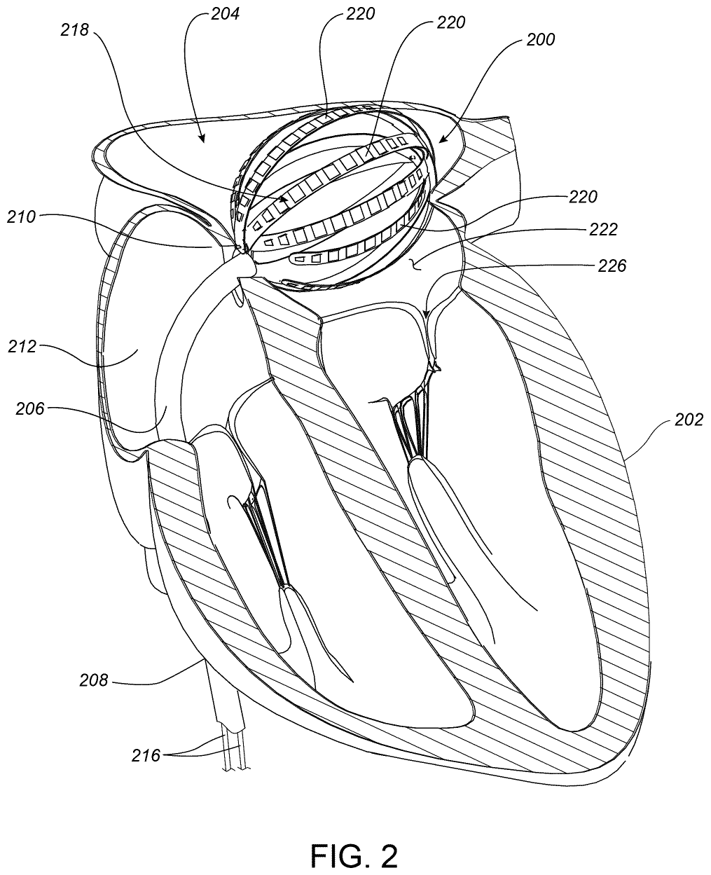

Various embodiments of transducer-based devices (e.g., forming part of catheters) are described herein in this disclosure. Some of the described devices are tissue ablation (e.g., PFA) devices that are percutaneously or intravascularly deployed. Some of the described devices are movable between a delivery or unexpanded configuration (e.g., FIG. 3A discussed below) in which a portion of the device is sized for passage through a bodily opening leading to a bodily cavity, and an expanded or deployed configuration (e.g., FIGS. 2 and 3B discussed below) in which the portion of the device has a size too large for passage through the bodily opening leading to the bodily cavity. An example of an expanded or deployed configuration, in some embodiments, is when the portion of the transducer-based device (e.g., catheter, or part thereof) is in its intended-deployed-operational state, which may be inside the bodily cavity when, e.g., performing an intended therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device (e.g., catheter, or part thereof) is being changed from the delivery configuration to the intended-deployed-operational state to a point where the portion of the device now has a size too large for passage through the bodily opening leading to the bodily cavity.

In some example embodiments, the described devices are part of a transducer-activation system capable of ablating tissue in a desired pattern within the bodily cavity using various techniques (e.g., via PFA, etc., according to various embodiments).

In some example embodiments, the devices are capable of sensing various cardiac functions (e.g., electrophysiological activity including intracardiac voltages which form the basis of recorded electrograms, according to some embodiments). In some example embodiments, the devices are capable of providing stimulation (e.g., electrical stimulation) to tissue within the bodily cavity. Electrical stimulation may include pacing.

FIG. 2 is a representation of a transducer-based device 200 useful in investigating or treating a bodily organ, for example, a heart 202, according to at least one example embodiment.

Transducer-based device 200 can be percutaneously or intravascularly inserted into a portion of the heart 202, such as an intra-cardiac cavity like left atrium 204. In this example, the transducer-based device 200 is part of a catheter 206 inserted via the inferior vena cava 208 and penetrating through a bodily opening in transatrial septum 210 from right atrium 212. (In this regard, transducer-based devices or device systems described herein that include a catheter may also be referred to as catheters, catheter devices or catheter-based devices, in some embodiments). In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 that are accessible externally from a patient in which the transducer-based device 200 is inserted.

According to some embodiments, transducer-based device 200 includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of a device 200 within, or with respect to left atrium 204. For example, transducers 220 may be used to determine a location of pulmonary vein ostia or a mitral valve 226, or both. In some embodiments, at least some of the transducers 220 may be used to selectively ablate portions of the tissue 222. In some embodiments, at least some of the transducers 220 are used to sense a physical characteristic of a fluid (e.g., blood) or tissue 222, or both, that may be used to determine contact or a degree of contact between a portion of a device 200 and a tissue surface (e.g., an internal tissue surface). In some embodiments, some of the transducers 220 may be used to ablate a pattern around the bodily openings, ports or pulmonary vein ostia, for instance to reduce or eliminate the occurrence of atrial fibrillation. In some embodiments, at least some of the transducers 220 are used to ablate cardiac tissue. In some embodiments, at least some of the transducers 220 are used to sense or sample intra-cardiac voltage data or sense or sample intra-cardiac electrogram data.

FIGS. 3A, 3B, and 3C (collectively, FIG. 3) include a transducer-based device system (e.g., a portion thereof shown schematically) that includes a catheter including a transducer-based device 300 according to some embodiments. Transducer-based device 300 includes a plurality of elongate members 304 (not all of the elongate members called out in each of FIGS. 3A, 3B, and 3C) and a plurality of transducers 306 (not all of the transducers called out in FIG. 3). FIG. 3C includes a representation of a portion of the transducer-based device 300 shown in FIG. 3B, but as viewed from a different viewing direction. It is noted that for clarity of illustration, all the elongate members shown in FIGS. 3B and 3C are not represented in FIG. 3A. The plurality of transducers 306 are positionable within a bodily cavity. For example, in some embodiments, the transducers 306 are able to be positioned in a bodily cavity by movement into, within, or into and within the bodily cavity, with or without a change in a configuration of the plurality of transducers 306. In some embodiments, the plurality of transducers 306 are arrangeable into various spatial distributions including two- or three-dimensional distributions, grids or arrays of the transducers capable of mapping, ablating or stimulating an inside surface of a bodily cavity or lumen. As shown, for example, in FIG. 3A, the plurality of transducers 306 are arranged in a configuration that is receivable in a bodily cavity. In various ones of FIGS. 3, each of at least some of transducers 306 includes a respective electrode 315 (not all of the transducers 306 or electrodes 315 are called out in each of the FIG. 3). According to various embodiments, the input-output device system 120 is communicatively connected to each electrode in the spatial distribution of electrodes 315 provided by the catheter. In some embodiments, the data processing device system (e.g., 110, 310) is communicatively connected to the plurality of electrodes 315 via the input-output device system (e.g., 120, 320).

According to some embodiments, the elongate members 304 may be arranged in a frame or structure 308 that is selectively movable between an unexpanded or delivery configuration (e.g., as shown in FIG. 3A) and an expanded or deployed configuration (e.g., as shown in FIGS. 3B and 3C) that may be used during a positioning of the elongate members 304 against a tissue surface within the bodily cavity or during a positioning of the elongate members 304 in the vicinity of the tissue surface. At least the expanded or deployed configuration shown in FIGS. 3B and 3C is an example of a spatial distribution of the transducers 306 (e.g., a three-dimensional spatial distribution). In some embodiments, structure 308 has a size in the unexpanded or delivery configuration suitable for delivery through a bodily opening (e.g., via catheter sheath 312 (shown in FIG. 3A but removed from FIGS. 3B and 3C for clarity)) to the bodily cavity. At least in a state in which the structure 308 is in the expanded or deployed configuration, the structure 308 may be considered to have two opposing poles 341a and 341b, marked by the intersection with axis 342 extending through the structure 308 as shown in FIGS. 3B and 3C according to some embodiments. In some embodiments, at least some of the plurality of transducers 306 are circumferentially arranged, e.g., in successive ring-like arrangements, about each of the poles 341a and 341b according to some embodiments. Two such ring-like arrangements are illustrated, for example, as broken-line rings 343a and 343b in FIG. 3B and FIG. 3C, respectively. According to some embodiments, at least some of the plurality of transducers 306 are arranged in a plurality of groups of the transducers 306, the groups of transducers 306 arranged like lines of longitude (e.g., along respective elongate members 304) about the structure 308 between each of the poles 341a and 341b, according to some embodiments.

In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB)). The elongate members 304 may include a plurality of different material layers. Each of the elongate members 304 may include a plurality of different material layers. The structure 308 may include a shape memory material, for instance, Nitinol. The structure 308 can include a metallic material, for instance stainless steel, or non-metallic material, for instance polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity or the requirements for successful ablation of a desired pattern.

FIG. 4 is a schematic side elevation view of at least a portion of a transducer-based device 400 that includes a flexible circuit structure 401 that is employed to provide a plurality of transducers 406 (two called out) according to an example embodiment. In some embodiments, the flexible circuit structure 401 may form part of a structure (e.g., structure 308) that is selectively movable between a delivery configuration sized for percutaneous delivery and an expanded or deployed configuration sized too large for percutaneous delivery. In some embodiments, the flexible circuit structure 401 may be located on, or form at least part of, a structural component (e.g., elongate member 304) of a transducer-based device system.

The flexible circuit structure 401 can be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b, and 404c (collectively electrically conductive layers 404) that are interleaved with the flexible layers 403. In some embodiments, each of the electrically conductive layers 404 is patterned to form various electrically conductive elements. For example, electrically conductive layer 404a is patterned to form a respective electrode 415 of each of the transducers 406. Electrodes 415 have respective electrode edges 415-1 that form a periphery of an electrically conductive surface associated with the respective electrode 415. It is noted that other electrodes employed in other embodiments may have electrode edges arranged to form different electrode shapes (for example, as shown by electrode edges 315-1 in FIG. 3B).

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass though vias in flexible layers 403a and 403b to connect with electrodes 415. Although FIG. 4 shows flexible layer 403c as being a bottom-most layer, some embodiments may include one or more additional layers underneath flexible layer 403c, such as one or more structural layers such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver ablation energy (e.g., PFA energy) to various tissue structures within a bodily cavity (e.g., an intra-cardiac cavity or chamber). The energy delivered to the tissue structures may be sufficient for ablating portions of the tissue structures. Energy that is sufficient for tissue ablation may be dependent upon factors including transducer location, size, shape, relationship with respect to another transducer or a bodily cavity, material or lack thereof between transducers, et cetera.

In some embodiments, each electrode 415 is employed to sense or sample an electrical potential in the tissue proximate the electrode 415 typically at a different time than delivering PFA energy sufficient for tissue ablation. In some embodiments, each electrode 415 is employed to sense or sample intra-cardiac voltage data in the tissue proximate the electrode 415. In some embodiments, each electrode 415 is employed to sense or sample data in the tissue proximate the electrode 415 from which an electrogram may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, leads 410a are arranged to allow for a sampling of electrical voltage between resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

Referring to FIGS. 3A, 3B, and 3C transducer-based device 300 can communicate with, receive power from or be controlled by a transducer-activation system 322 (e.g., via leads 317). In some embodiments, the transducer-activation system 322 represents one or more particular implementations of the system 100 illustrated in FIG. 1. In some embodiments, the transducer-based device 300 or the transducer-based device 200 may be considered part of the transducer-activation system 322 or 100. However, the transducer activation system 322 or 100, according to various embodiments, is not limited to including or interacting with either of the particular transducer-based devices 200, 300, and may include or interact with one or more other types of transducer-based devices in some embodiments.

The transducer-activation device system 322 may include a controller 324 that includes a data processing device system 310 (which may be a particular implementation of data processing device system 110 from FIG. 1) and a memory device system 330 (which may be a particular implementation of the memory device system 130 from FIG. 1) that stores data and instructions that are executable by the data processing device system 310 to process information received from transducer-based device 300 or to control operation of transducer-based device 300, for example, activating various selected transducers 306 to ablate tissue (e.g., via PFA) according to various embodiments including at least those described below with respect to FIG. 5. Controller 324 may include one or more controllers.

Transducer-activation device system 322 includes an input-output device system 320 (which may be a particular implementation of the input-output device system 120 from FIG. 1) communicatively connected to the data processing device system 310 (e.g., via controller 324 in some embodiments). Input-output device system 320 may include a sensing device system 325 configured to detect various characteristics including, but not limited to, at least one of tissue characteristics (e.g., electrical characteristics such as tissue impedance, tissue conductivity, tissue type, tissue thickness) and thermal characteristics. In this regard, the sensing device system 325 may include one, some, or all of the transducers 306 (or 406 of FIG. 4) of the transducer-based device 300, including the internal components of such transducers shown in FIG. 4, such as the electrodes 415 and temperature sensors 408.

Transducer-activation device system 322 may also include an energy source device system 340 (also referred to as power supply system in some embodiments) including one or more energy source devices (e.g., one or more power delivery drivers 344 (two shown in FIGS. 3A-3C as 344a, 344b) in some embodiments) selectively connectable (e.g., via electrical switches) to transducers 306. In this regard, although various ones of FIG. 3 show a communicative connection between the energy source device system 340 and the controller 324 (and its data processing device system 310), the energy source device system 340 may also be connected (e.g., via electrical switches) to the transducers 306 via a communicative connection that is independent of the communicative connection between the energy source device system 340 and the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more electrical switches and communication lines through catheter body or shaft 314, elongated cable 316 or catheter sheath 312) that does not pass through the controller 324. In this regard, the energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 or electrodes thereof to selectively provide energy, e.g., via one or more power delivery drivers 344, in the form of electrical current or power (e.g., PFA energy) to cause ablation of tissue. In some embodiments, a power delivery driver may be a circuit used to deliver electrical power to a load. In some embodiments, the load may be a transducer (e.g., electrode). In some embodiments, the load may be tissue, such as tissue proximate a transducer. A power delivery driver may include a circuit that is controllable to produce a specified voltage output (e.g., high voltage pulses), in some embodiments. In some embodiments, a power delivery driver may include a circuit that is controllable to produce a specified current output (e.g., the power delivery driver may adjust its output voltage as required to achieve a specified current). The energy source device system 340 may selectively provide energy, e.g., via one or more power delivery drivers 344, in the form of electrical current to various selected transducers 306 or electrodes thereof and such transducers 306 or electrodes thereof may measure a temperature characteristic, an electrical characteristic, or both at a respective location at least proximate each of the various transducers 306 utilizing energy provided by the energy source device system 340. The energy source device system 340 may include various electrical current or voltage sources, such as power delivery drivers 344, as energy source devices.

It is understood that input-output device system 320 may include various systems. In some embodiments, input-output device system 320 may include energy source device system 340, transducer-based device 300, or both energy source device system 340 and transducer-based device 300 by way of non-limiting example. Input-output device system 320 may include the memory device system 330 in some embodiments.

In other example embodiments, other structures besides those shown in FIGS. 2, 3A, 3B, 3C, and 4 may be employed to support or carry transducers of a transducer-based device, such as a transducer-based catheter. For example, an elongated catheter member may be used to distribute the transducers in a linear or curvilinear array. Basket catheters or balloon catheters may be used to distribute the transducers in a two-dimensional or three-dimensional array.

According to some embodiments of the present invention, the system 100 (FIG. 1) includes some, or all, of the system 200 shown in FIG. 2, or vice versa. In some embodiments, the system 100 includes some, or all, of the system 300 in FIG. 3, or vice versa. In this regard, the system 200, the system 300, or each of the system 200 and the system 300 may be a particular implementation of the system 100, according to some embodiments. Some or all of the controller 324, energy source device system 340, or input-output device system 320 described with respect to FIG. 3 may also be implemented with the system 200 in FIG. 2, in some embodiments. Each of at least part of the transducer or electrode-based device system 400 in FIG. 4 may be part of the system 100, the system 200, or the system 300, according to various embodiments.

FIG. 5 includes a processing flow diagram, which may implement various embodiments of method 500 by way of associated computer-executable instructions according to some example embodiments. In various example embodiments, a memory device system (e.g., memory device system 130 or 330) is communicatively connected to a data processing device system (e.g., data processing device systems 110 or 310, otherwise stated herein at times as “110, 310”) and stores a program executable by the data processing device system to cause the data processing device system to execute various embodiments of method 500 via interaction with at least, for example, a transducer-based or electrode-based device (e.g., transducer-based/electrode-based devices 200, 300, or 400). In these various embodiments, the program may include instructions configured to perform, or to cause to be performed, various ones of the instructions associated with execution of various embodiments of method 500. In some embodiments, method 500 may include a subset of the associated blocks or additional blocks than those shown in FIG. 5. In some embodiments, method 500 may include a different sequence indicated between various ones of the associated blocks shown in FIG. 5.

According to some embodiments, method 500 may include block 502 associated with computer-executable instructions (e.g., first energy delivery instructions provided by a program) configured to cause a data processing device system (e.g., 110, 310) to cause, via an input-output device system (e.g., input-output device system 120, 320 or energy source device system 340), a first group of electrodes from a plurality of electrodes (e.g., 315, 415) to concurrently and collectively deliver as a group, first energy configured to cause pulsed field ablation of bodily tissue. The first group of electrodes may be a group of electrodes selected to cause ablation of tissue during a medical procedure for treatment of a medical condition. In some embodiments, the first group of electrodes has an even number of electrodes. Such a configuration may be beneficial at least in some contexts where bipolar ablation is desired, where the even number of electrodes can be divided into two sub-groups of electrodes of equal numbers of electrodes, where, if the two sub-groups of electrodes have opposite polarities during the bipolar ablation, energy flow can be relatively balanced between the two sub-groups during the bipolar ablation. Similarly, each electrode includes a respective energy transmission surface configured to transmit tissue-ablative energy, and, in some embodiments, the first group of electrodes includes two sub-groups of electrodes, where each sub-group has roughly an equivalent (e.g., within 10% in some embodiments) total combined energy transmission surface area as the other sub-group. Such a configuration may be helpful at least in some contexts where bipolar ablation is desired, where, if the two sub-groups of electrodes have opposite polarities during the bipolar ablation, energy flow can be balanced between the two sub-groups due to the roughly equivalent total combined energy transmission surface areas.

In some embodiments, the first group of electrodes includes at least four electrodes or at least eight electrodes. In this regard, it may be beneficial to have a relatively large number of electrodes in the first group of electrodes in an attempt to reduce overall procedure time. In the example of FIGS. 3B and 3C, the first group of electrodes is identified, for example, with a hatch pattern and references 315a1, 315a2, 315a3, 315a4, 315b1, 315b2, 315b3, and 315b4 (collectively referred to with reference 315G). The selection of the first group of electrodes may occur in any manner known in the art, e.g., by user selection via a user interface, by machine selection by the data processing device system (e.g., 110, 310) based, e.g., on sensing input from one or more electrodes (e.g., 315 or other sensing device), or by user and machine selection, according to some embodiments. For instance, a user interface may be presented to a physician via a display device system 332 of input-output device system 320 in FIG. 3B and FIG. 3C, where the physician operates an input device, such as mouse 335 to click on graphical representations of the electrodes corresponding to the first group of electrodes 315G in order to select such electrodes for tissue ablation. However, any manner of identifying the first group of electrodes may be utilized.

In some embodiments, block 502 may be associated with computer-executable instructions configured to cause the data processing device system (e.g., 110, 310) to cause an energy source device system (e.g., 340) to transmit the first energy concurrently and collectively to the first group of electrodes (e.g., 315G) so that the first group of electrodes may deliver such first energy to target tissue in order to ablate such tissue. In this regard, the first energy may be considered a collective energy delivered by the entire first group of electrodes.

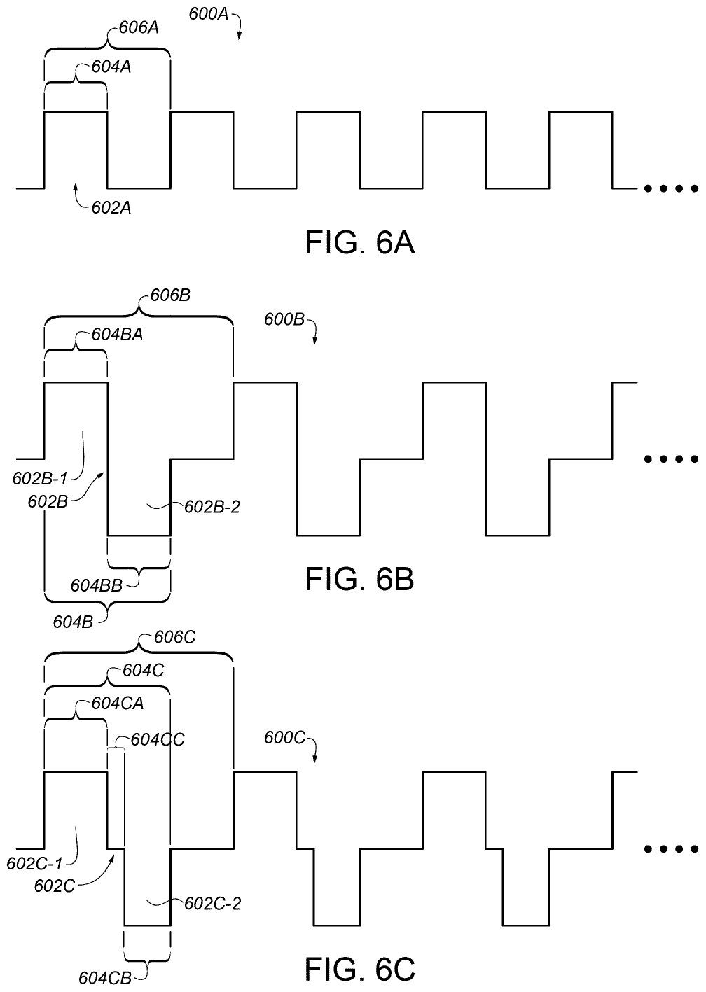

In some embodiments, the first energy is a train of voltage pulses, also referred to in some embodiments as a voltage pulse train or simply a pulse train. In some embodiments, the pulse train is configured to cause PFA of target tissue. Various pulse train configurations may be employed for the first energy (and for the second energy described below with respect to at least block 506 in FIG. 5), according to various embodiments. For example, FIG. 6A shows a portion of a pulse train 600A, according to some embodiments. It is noted that the waveforms shown in FIG. 6A, as well as FIG. 6B and FIG. 6C discussed in more detail below, may not be to scale and are merely presented for purposes of illustration. For example, the width (e.g., width 604A) of each pulse shown in FIG. 6A might represent a much smaller fraction of the period (e.g., period 606A) of the respective pulse than that shown in FIG. 6A, in some embodiments. According to various embodiments, pulse train 600A includes a plurality of high voltage pulses 602A (only one called out). According to various embodiments, each of the high voltage pulses 602A is a monophasic pulse with each pulse 602A having a same polarity. According to some embodiments, each high voltage pulse 602A has a pulse width 604A. According to various embodiments, the high voltage pulses 602A repeat in pulse train 600A according to period 606A.

FIG. 6B shows a portion of a pulse train 600B according to various embodiments. According to various embodiments, pulse train 600B includes a plurality of high voltage pulses 602B (only one called out). According to some embodiments, each high voltage pulse 602B is a biphasic pulse. For example, in FIG. 6B, each high voltage pulse 602B has a first pulse portion 602B-1 and a second pulse portion 602B-2, the second pulse portion 602B-2 having a different polarity than the polarity of the first pulse portion 602B-1. According to some embodiments, each high voltage pulse 602B has a pulse width 604B. According to various embodiments, pulse width 604B is determined at least by a pulse width 604BA of the first pulse portion 602B-1 and a pulse width 604BB of the second pulse portion 602B-2. According to various embodiments, the high voltage pulses 602B repeat in pulse train 600B according to period 606B.

FIG. 6C shows a portion of a pulse train 600C according to various embodiments. According to some embodiments, pulse train 600C includes a plurality of high voltage pulses 602C (only one called out). According to some embodiments, each high voltage pulse 602C is a biphasic pulse. For example, in FIG. 6C, each high voltage pulse 602C has a first pulse portion 602C-1 and a second pulse portion 602C-2, the second pulse portion 602C-2 having a different polarity than the polarity of the first pulse portion 602C-1. Unlike the biphasic pulses 602B shown in FIG. 6B, the biphasic pulses 602C include an inter-phase gap 604CC between the first pulse portion 602C-1 and the second pulse portion 602C-2. The inter-phase gap 604CC may be motivated for different reasons. For example, in some cardiac ablation procedures a relatively small inter-phase gap 604CC may lead to less muscle twitching while a relatively large inter-phase gap 604CC may be more effective in forming lesions. According to some embodiments, each high voltage pulse 602C has a pulse width 604C. According to various embodiments, pulse width 604C is determined at least by a pulse width 604CA of the first pulse portion 602C-1, a pulse width 604CB of the second pulse portion 602C-2, and the inter-phase gap 604CC. Although FIG. 6C shows different durations for pulse width 604CA and pulse width 604CB, such pulse widths may instead have the same duration in some embodiments. According to various embodiments, the high voltage pulses 602C (encompassing pulse width 604C) repeat in pulse train 600C according to period 606C.

The choice of particular monophasic pulses or biphasic pulses in a particular PFA procedure may be motivated by different reasons and may vary in different applications. Possible advantages of monophasic pulses may include typically more efficient cellular damage per pulse (e.g., the charge applied to the cell membrane is not undone with a subsequent phase change) and the possibility of added therapeutic effect by the interaction of a pH front with the electroporation effects, resulting in deeper lesions or requiring fewer pulses to achieve lesions of a certain depth. Possible advantages of biphasic pulses may include reductions in muscle contractions, nerve stimulation, and microbubble formation.

In some embodiments, the first energy may be predefined or predetermined (e.g., as recorded in memory device system 130, 330) to deliver a set number of voltage pulses over a predefined or predetermined period of time.

In some embodiments, the energy source device system (e.g., 340) includes a plurality of power delivery drivers (e.g., 344, two shown as 344a, 344b in FIGS. 3A-3C) configured to transmit energy to the plurality of electrodes. In some embodiments in which the energy source device system includes only a single power delivery driver (e.g., such as only power delivery driver 344a in FIGS. 3A-3C), such single power delivery driver is configured to transmit the first energy to the first group of electrodes (e.g., 315G) for delivery to the target tissue by such first group of electrodes. In some embodiments in which the energy source device system includes a plurality of power delivery drivers (e.g., such as at least power delivery drivers 344a, 344b shown in FIGS. 3A-3C), a single power delivery driver (which may be referred to as “one power delivery driver” of the plurality of power delivery drivers in some embodiments) thereof may be utilized to transmit the first energy to the first group of electrodes. Such a configuration may be useful at least in some contexts in which the first group of electrodes (e.g., 315G) is connected or coupled to at least such single power delivery driver. In view of the above, for example, the causing the first group of electrodes from the plurality of electrodes to concurrently and collectively deliver as a group the first energy configured to cause pulsed field ablation of bodily tissue per block 502 may include, in some embodiments, causing the single power delivery driver (or one power delivery driver) to transmit the first energy to the first group of electrodes. In some embodiments in which the energy source device system includes a plurality of power delivery drivers, the causing the first group of electrodes from the plurality of electrodes to concurrently and collectively deliver as a group the first energy configured to cause pulsed field ablation of bodily tissue per block 502 may include causing more than one or all of the plurality of power delivery drivers to transmit the first energy to the first group of electrodes. Such configurations may be useful at least in some contexts in which the first group electrodes is connected or coupled to such more than one or all of the power delivery drivers.

Configuring the energy source device system (e.g., 340) to have one or more power delivery drivers (e.g., 344) may depend on the needs of a particular medical procedure. For instance, having a single power delivery driver may reduce cost of the energy source device system, but may reduce redundancy in the energy source device system as compared to a multiple power delivery driver configuration. On the other hand, a multiple power delivery driver configuration may increase cost of the energy source device system and may increase complexity of electrically connecting the electrodes 315 to the energy source device system, but may enhance redundancy.

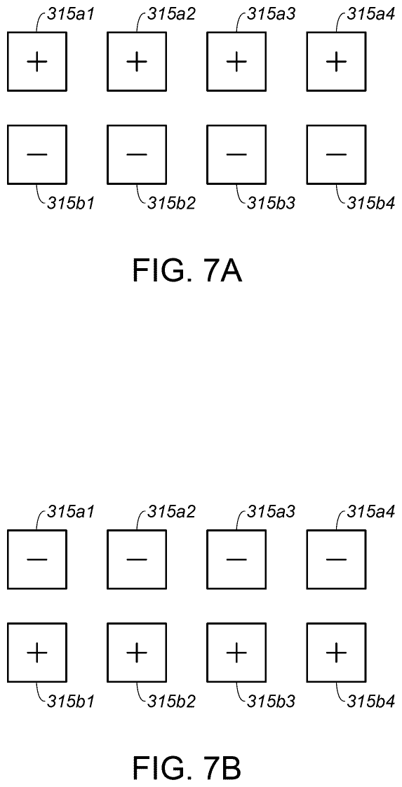

FIGS. 7A and 7B illustrate an example of the concurrent and collective delivery of the first energy by the first group of electrodes (e.g., 315G) per block 502, according to some embodiments. In this regard, each of FIGS. 7A and 7B is a simplified representation of the first group of electrodes 315G (i.e., electrodes 315a1, 315a2, 315a3, 315a4, 315b1, 315b2, 315b3, and 315b4). The example of FIGS. 7A and 7B assumes a biphasic voltage waveform being applied to the first group of electrodes 315G by the energy source device system 340, according to some embodiments. In some embodiments, such a biphasic waveform may be a sequence of biphasic voltage pulse sets, where, in some embodiments, each biphasic voltage pulse set may include or be one or more biphasic voltage pulses.

In this regard, FIG. 7A may represent a state in which all electrodes of the first group of electrodes 315G are concurrently and collectively connected as a group (e.g., by one or more electrical switches in a closed state) to the energy source device system 340 (or one or more power delivery drivers 344 thereof), according to some embodiments. In the example in FIG. 7A, electrodes 315a1, 315a2, 315a3, and 315a4 concurrently have a positive polarity for the energy provided from the energy source device system 340 (or one or more power delivery drivers 344 thereof), while concurrently, electrodes 315b1, 315b2, 315b3, and 315b4 concurrently have a negative polarity for the energy provided from the energy source device system 340 (or one or more power delivery drivers 344 thereof), such that current flows from the anodes (i.e., electrodes 315a1, 315a2, 315a3, and 315a4) to the cathodes (i.e., electrodes 315b1, 315b2, 315b3, and 315b4).

After the state of FIG. 7A, the state represented by FIG. 7B may occur, where the polarities of the electrodes in the first group of electrodes 315G are flipped compared to the state of FIG. 7A. For instance, the energy source device system 340 may include, in some embodiments, an H-bridge, known in the art, on a transformer primary that reverses the polarity of the voltage applied to the transformer primary (i.e., such that a first driver output pole is positive with respect to a second driver output pole in the first phase, and the opposite in the second phase), according to some embodiments. The example of FIG. 7B illustrates the flipped polarities compared to FIG. 7A. In particular, in the state of FIG. 7B, electrodes 315a1, 315a2, 315a3, and 315a4 concurrently have a negative polarity for the energy provided from the energy source device system 340 (or one or more power delivery drivers 344 thereof), while concurrently, electrodes 315b1, 315b2, 315b3, and 315b4 concurrently have a positive polarity for the energy provided from the energy source device system 340 (or one or more power delivery drivers 344 thereof), such that current flows from the anodes (i.e., electrodes 315b1, 315b2, 315b3, and 315b4) to the cathodes (i.e., electrodes 315a1, 315a2, 315a3, and 315a4).

Accordingly, in some embodiments, the sequence of the states represented by FIGS. 7A and 7B may represent the delivery of a biphasic voltage pulse, where the state of FIG. 7A may represent the delivery of a first phase of the biphasic voltage pulse, and the state of FIG. 7B may represent the delivery of a second phase of the biphasic voltage pulse, the second phase having an opposite polarity configuration compared to the first phase. In some embodiments, the states represented by FIGS. 7A and 7B may be cycled or repeated to produce a plurality of biphasic voltage pulses at least as part of the delivery of the first energy by the first group of electrodes per block 502, according to some embodiments.

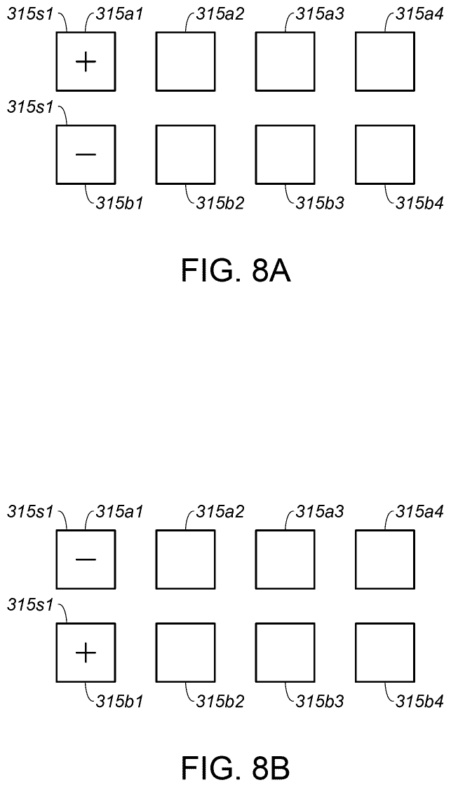

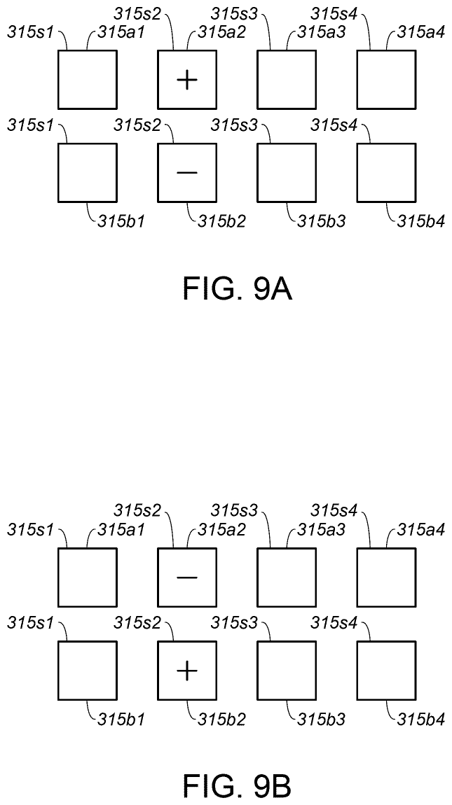

Although the examples of FIGS. 7A and 7B, as well as the examples of at least FIGS. 8A, 8B, 9A, and 9B discussed below, show opposing polarities being delivered to adjacent electrodes on the same elongate member 304 per, e.g., at least FIG. 3C, other embodiments may instead require that electrodes along the same elongate member 304 have the same polarity, such that opposing polarities, e.g., for a biphasic voltage waveform, are applied to adjacent electrodes on adjacent elongate members 304 instead of along the same elongate member. Other embodiments may apply opposing polarities to adjacent electrodes either along a same elongate member 304 or between adjacent elongate members.

At least in some contexts or use cases, it may be advantageous to transmit the first energy concurrently and collectively (per block 502) to the first group of electrodes (e.g., 315G) in order to reduce procedure time compared to transmitting such energy non-concurrently to subsets of electrodes that form the first group of electrodes. However, the present inventors recognized that if a fault condition arises during the attempted delivery of the first energy concurrently and collectively by the first group of electrodes, it can be more difficult to determine specifically where the fault condition exists among all electrodes in the first group of electrodes compared to a case where energy is delivered to a smaller number of electrodes. On the other hand, delivering energy to smaller numbers of electrodes sequentially has the effect of increasing overall procedure time as compared to the case where energy is delivered to a larger group of electrodes concurrently and collectively. Accordingly, some embodiments of the present invention seek to merge the benefits of both cases by first attempting to deliver the ablative energy to the first group of electrodes (e.g., per block 502), but if a fault condition is detected, then energy is attempted to be delivered non-concurrently to subsets of electrodes making up the first group of electrodes.

In this regard, according to some embodiments, method 500 may include block 504 associated with computer-executable instructions (e.g., identification instructions or first identification instructions provided by a program) configured to cause a data processing device system (e.g., 110, 310) to identify, at least in response to the first group of electrodes (e.g., 315G) concurrently and collectively attempting to deliver as a group the first energy (per block 502) to cause pulsed field ablation of bodily tissue, that a fault condition has occurred. According to some embodiments, the fault condition may be a dielectric breakdown, an overcurrent or low impedance condition, or a low current or high impedance condition. For instance, a dielectric breakdown may be a breakdown of the dielectric in the electrical circuit path between the energy source device system (e.g., 340) and one or more of the electrodes in the first group of electrodes (e.g., 315G) due, e.g., to a material failure, a dielectric breakdown of blood or tissue, or short-circuit condition revealed by transmission or delivery of the first energy. In this regard, a short-circuit condition also may produce an overcurrent or low impedance condition. On the other hand, a low current or high impedance condition may also be identified as the fault condition, for instance, in the case of an open-circuit, or a case in which a high impedance obstruction (e.g., an open circuit failure) is located between an electrode of the first group of electrodes and target tissue, or, for instance, in a case in which there is a failure in the energy source device system preventing transmission of the full current necessary for the first energy, according to some embodiments.

In some embodiments, the data processing device system (e.g., 110, 310) is configured by program instructions to identify that the fault condition has occurred by analyzing information from one or more sensors. For instance, in some embodiments, method 500 may include block 503 associated with computer-executable instructions (e.g., reception instructions provided by a program) configured to cause the data processing device system (e.g., 110, 310) to receive, via the input-output device system (e.g., 120, 320), a signal set. In this regard, in some embodiments, the data processing device system may be configured by the program at least to execute the identifying (per block 504) that the fault condition has occurred based at least on an analysis of the received signal set. Block 503 in FIG. 5 is shown in broken line to accentuate that it is an optional part of method 500 in some embodiments.

In some embodiments, the signal set is received by the data processing device system from one or more sensors via the input-output device system. In some embodiments, the one or more sensors is an electrode set. In this regard, in some embodiments, the data processing device system may be configured by the program at least to receive, via the input-output device system, the signal set from an electrode set. In some embodiments, the electrode set may be from the plurality of electrodes 315 shown in FIGS. 3A-C at least in some embodiments in which the electrode set is configured to perform a sensing function in addition to an ablation function. In this regard, in some embodiments, the electrode set may be from the first group of electrodes (e.g., 315G) at least in some embodiments in which the electrode set is configured to perform a sensing function while performing the ablation function.

In some embodiments, the signal set provided (e.g., per block 503) by the one or more sensors or electrode set to the data processing device system (e.g., 110, 310) for analysis may include results of the one or more sensors or electrode set sensing or monitoring current flow during the attempted delivery of the first energy. The data processing device system may analyze such results to determine if the sensed current flow exceeds an expected or threshold value and, if so, the data processing device system may then determine or identify that the fault condition has occurred.

In view of the above, it can be seen that some embodiments of the present invention attempt to first deliver tissue-ablative energy collectively to the first group of electrodes (e.g., 315G) per block 502 and then monitor (e.g., by analyzing the received signal set per block 503 in some embodiments) the delivery of such tissue-ablative energy for occurrence of a fault condition. If a fault condition is identified per block 504, then delivery of tissue-ablative energy to subsets of electrodes that make up the first group of electrodes is attempted in some embodiments. Such delivery can, in some embodiments, help isolate the electrode or electrodes in the first group of electrodes that is or are involved in the fault condition, while contemporaneously allowing the medical procedure to safely proceed by delivering tissue-ablative energy to electrodes in the first group of electrodes that are not involved in the fault condition, thereby reducing medical procedure duration as compared to a case where the fault condition merely stops the medical procedure in its entirety.

In this regard, according to some embodiments, method 500 may include block 506 associated with computer-executable instructions (e.g., second energy delivery instructions provided by a program) configured to cause the data processing device system (e.g., 110, 310) to cause, via the input-output device system (e.g., input-output device system 120, 320 or energy source device system 340) and in response to identifying (per block 504) that the fault condition has occurred, the first group of electrodes (e.g., 315G) to non-concurrently deliver, in separate subsets of electrodes, respective second energies to cause pulsed field ablation of bodily tissue. In this regard, in some embodiments, each of the separate subsets of electrodes receives its own respective second energy configured to cause tissue ablation according to block 506. The separate subsets of electrodes may include the electrodes that collectively make up the first group of electrodes, in some embodiments. Accordingly, in some embodiments, each of the separate subsets of electrodes includes less than all of the electrodes that make up the first group of electrodes.

In some embodiments, each of the separate subsets of electrodes includes two or more electrodes. In some embodiments, each of the separate subsets of electrodes has an even number of electrodes. In some embodiments, each of the separate subsets of electrodes is a pair of electrodes. In some embodiments, each of one or more or all of the separate subsets of electrodes includes at least one electrode that is also in at least one other of the separate subsets of electrodes. In some embodiments, each of one or more or all of the separate subsets of electrodes has at least one electrode that is other than another electrode in another of the separate subsets of electrodes. In some embodiments, each of one or more or all of the separate subsets of electrodes has at least one electrode that is not in any other of the separate subsets of electrodes. In some embodiments, each of the separate subsets of electrodes does not include any electrode that is also included in any of the other separate subsets of electrodes. In some embodiments, each of one or more or all of the separate subsets of electrodes has a different number of electrodes than each of at least one or more or every other of the separate subsets of electrodes.

In some embodiments in which the energy source device system (e.g., 340) includes only a single power delivery driver (e.g., such as only power delivery driver 344a in FIGS. 3A-3C), such single power delivery driver may be utilized to transmit all of the respective second energies to the separate subsets of electrodes for block 506. In some embodiments in which the energy source device system includes a plurality of power delivery drivers (e.g., such as at least power delivery drivers 344a, 344b shown in FIGS. 3A-3C), a single power delivery driver thereof may be utilized to transmit all of the respective second energies to the separate subsets of electrodes for block 506. Such a configuration may be useful at least in some contexts in which the separate subsets of electrodes is connected or coupled to at least such single power delivery driver. In view of the above, for example, the causing the first group of electrodes to non-concurrently deliver, in the separate subsets of electrodes, the respective second energies to cause pulsed field ablation of bodily tissue per block 506 may include, in some embodiments, causing the single power delivery driver to transmit the respective second energies to the separate subsets of electrodes.

In some embodiments in which the energy source device system (e.g., 340) includes a plurality of power delivery drivers (e.g., such as at least power delivery drivers 344a, 344b in FIGS. 3A-3C), different ones of the power delivery drivers may be utilized to deliver different ones of the respective second energies to different ones of the separate subsets of electrodes for block 506. For instance, in some embodiments, block 506 may be associated with computer-executable instructions configured to cause the data processing device system (e.g., 110, 310) to cause, via the input-output device system (e.g., input-output device system 120, 320 or energy source device system 340), a first power delivery driver (e.g., 344a) of the plurality of power delivery drivers to transmit a respective one of the respective second energies to a first subset of the separate subsets of electrodes, and to cause a second power delivery driver (e.g., 344b) of the plurality of power delivery drivers to transmit a respective one of the respective second energies to a second subset of the separate subsets of electrodes.

In some embodiments, each of one or more or all of the respective second energies delivered to the separate subsets of electrodes, respectively, per block 506 may be configured the same or have the same composition or waveform as the first energy as discussed above with respect to block 502. In this regard, the above discussions set forth with respect to FIGS. 6A-6C apply as well to one or more or all of the respective second energy delivered according to block 506. However, in some embodiments, each of one or more or all of the respective second energies delivered to the separate subsets of electrodes, respectively, per block 506, may have a different configuration or composition or waveform than the first energy.

FIGS. 8A, 8B, 9A, and 9B illustrate an example of the non-concurrent delivery of two respective second energies by two respective separate subsets 315s1, 315s2 of electrodes per block 506, according to some embodiments. In this regard, each of FIGS. 8A, 8B, 9A, and 9B is a simplified representation of the first group of electrodes 315G (i.e., electrodes 315a1, 315a2, 315a3, 315a4, 315b1, 315b2, 315b3, and 315b4). The example of FIGS. 8A, 8B, and the example of FIGS. 9A and 9B assume a biphasic voltage waveform being applied (e.g., by one or more signals) to the two separate subsets 315s1 (in the example of FIGS. 8A and 8B) and 315s2 (in the example of FIGS. 9A and 9B) of electrodes by the energy source device system 340, according to some embodiments. In some embodiments, such biphasic waveforms may each be a sequence of biphasic voltage pulse sets, where, in some embodiments, each biphasic voltage pulse set may include or be one or more biphasic voltage pulses.

Although the examples of FIGS. 8A, 8B, 9A, and 9B specifically discuss two of the separate subsets of electrodes to explain how all of the separate subsets of electrodes may be caused to behave, all of the separate subsets would make up all of the electrodes in the first group of electrodes 315G, according to some embodiments. In particular, the first subset 315s1 of electrodes discussed in more detail below with respect to FIGS. 8A and 8B includes electrodes 315al and 315b1, and the second subset 315s2 of electrodes discussed in more detail below with respect to FIGS. 9A and 9B includes electrodes 315a2 and 315b2. However, the remaining subsets of electrodes in this example may be a subset 315s3 of electrodes 315a3, 315b3, and a subset 315s4 of electrodes 315a4, 315b4, such that all of these subsets collectively make up all of the electrodes in the first group of electrodes 315G, according to some embodiments.

FIG. 8A may represent a state in which all electrodes of a first subset 315s1 of electrodes 315G are concurrently and collectively connected as a group (e.g., by one or more electrical switches in a closed state) to the energy source device system 340 (or a power delivery driver 344 thereof), according to some embodiments, to deliver a respective one of the second energies to the first subset 315s1 of electrodes per an example implementation of block 506. In the example in FIG. 8A, electrode 315al has a positive polarity for the energy provided from the energy source device system 340 (or, e.g., a power delivery driver 344a thereof), while concurrently, electrode 315b1 has a negative polarity for the energy provided from the energy source device system 340 (or, e.g., power delivery driver 344a thereof), such that current flows from the anode (i.e., electrode 315a1) to the cathode (i.e., electrode 315b1).

After the state of FIG. 8A, the state represented by FIG. 8B may occur, where the polarities of the electrodes in the first subset 315s1 of electrodes are flipped compared to the state of FIG. 8A. In this regard, in the example of FIG. 8B, electrode 315al has a negative polarity for the energy provided from the energy source device system 340 (e.g., power delivery driver 344a thereof), while concurrently, electrode 315b1 has a positive polarity for the energy provided from the energy source device system 340 (e.g., power delivery driver 344a thereof), such that current flows from the anode (i.e., electrode 315b1) to the cathode (i.e., electrode 315a1).

Accordingly, in some embodiments, the sequence of the states represented by FIGS. 8A and 8B may represent the delivery of a biphasic voltage pulse as part of the delivery of a respective second energy to the first subset 315s1 of electrodes per an example implementation of block 506, where the state of FIG. 8A may represent the delivery of a first phase of the biphasic voltage pulse, and the state of FIG. 8B may represent the delivery of a second phase of the biphasic voltage pulse, the second phase having an opposite polarity configuration compared to the first phase.

After the state of FIG. 8B, the state represented by FIG. 9A may occur. FIG. 9A may represent a state in which all electrodes of a second subset 315s2 of electrodes 315G are concurrently and collectively connected as a group (e.g., by one or more electrical switches in a closed state) to the energy source device system 340 (or a power delivery driver 344 thereof), according to some embodiments, to deliver a respective one of the second energies to the second subset 315s2 of electrodes per an example implementation of block 506. In the example in FIG. 9A, electrode 315a2 has a positive polarity for the energy provided from the energy source device system 340 (or, e.g., a power delivery driver 344a or 344b (if multiple power delivery drivers are present) thereof), while concurrently, electrode 315b2 has a negative polarity for the energy provided from the energy source device system 340 (or, e.g., power delivery driver 344a or 344b thereof), such that current flows from the anode (i.e., electrode 315a2) to the cathode (i.e., electrode 315b2).

After the state of FIG. 9A, the state represented by FIG. 9B may occur, where the polarities of the electrodes in the second subset 315s2 of electrodes are flipped compared to the state of FIG. 9A. In this regard, in the example of FIG. 9B, electrode 315a2 has a negative polarity for the energy provided from the energy source device system 340 (e.g., power delivery driver 344a or 344b thereof), while concurrently, electrode 315b2 has a positive polarity for the energy provided from the energy source device system 340 (e.g., power delivery driver 344a or 344b thereof), such that current flows from the anode (i.e., electrode 315b2) to the cathode (i.e., electrode 315a2).

Accordingly, in some embodiments, the sequence of the states represented by FIGS. 9A and 9B may represent the delivery of a biphasic voltage pulse as part of the delivery of a respective second energy to the second subset 315s2 of electrodes per an example implementation of block 506, where the state of FIG. 9A may represent the delivery of a first phase of the biphasic voltage pulse, and the state of FIG. 9B may represent the delivery of a second phase of the biphasic voltage pulse, the second phase having an opposite polarity configuration compared to the first phase.

In some embodiments, the sequence of events described above for the first subset 315s1 of electrodes and the second subset 315s2 of electrodes may be continued for each of the remaining subsets of electrodes in this example, namely, the subset 315s3 of electrodes 315a3, 315b3, and the subset 315s4 of electrodes 315a4, 315b4, such that a biphasic voltage pulse is delivered to each of the subsets of electrodes in turn. Then, according to some embodiments, this process for all of the separate subsets of electrodes may be repeated, such that a cycling of delivery of biphasic voltage pulses through the separate subsets of electrodes may occur in order to deliver the respective second energies pursuant to an example implementation of block 506 in FIG. 5.

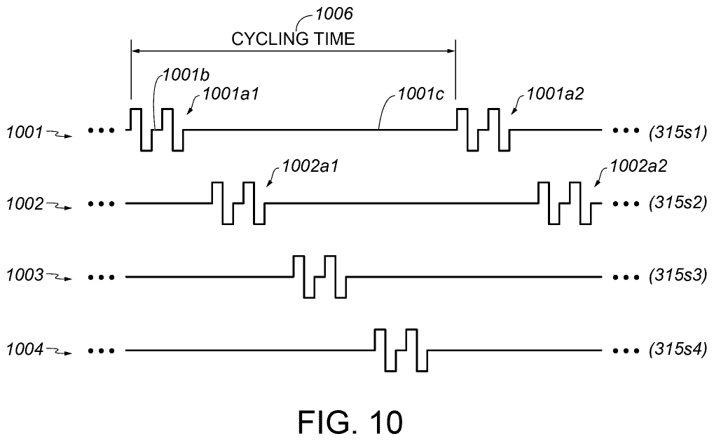

FIG. 10 illustrates this cycling example in greater detail. It is noted that the waveforms shown in FIG. 10 may not be to scale and are merely presented for purposes of illustration. In this regard, FIG. 10 illustrates a cycling of a sequence of voltage waveforms 1001, 1002, 1003, and 1004 respectively applied to the separate subsets 315s1, 315s2, 315s3, and 315s4 of electrodes. The horizontal axes of the voltage waveforms shown in FIG. 10 represent time, and the vertical axes of the voltage waveforms shown in FIG. 10 represent voltage. Subset 315s1 corresponds to electrodes 315al and 315b1, subset 315s2 corresponds to electrodes 315a2 and 315b2, subset 315s3 corresponds to electrodes 315a3 and 315b3, and subset 315s4 corresponds to electrodes 315a4 and 315b4, in this example. Accordingly, voltage waveform 1001 is transmitted by the energy source device system 340 (or a power delivery driver thereof) to subset 315s1 of electrodes, voltage waveform 1002 is transmitted by the energy source device system 340 (or a power delivery driver thereof) to subset 315s2 of electrodes, voltage waveform 1003 is transmitted by the energy source device system 340 (or a power delivery driver thereof) to subset 315s3 of electrodes, and voltage waveform 1004 is transmitted by the energy source device system 340 (or a power delivery driver thereof) to subset 315s4 of electrodes, in this example pursuant to block 506 in FIG. 5.

Each of the voltage waveforms (such as, e.g., voltage waveforms 1001-1004 in some embodiments) applied to the respective subsets of electrodes to deliver the respective second energies may be a train of voltage pulses, a train of voltage pulse sets, or a sequence of voltage pulse sets, according to various embodiments. In the example of FIG. 10, only a first voltage pulse set 1001a1 and a second voltage pulse set 1001a2 delivered by the first subset of electrodes 315s1 are shown for voltage waveform 1001, although additional voltage pulse sets may be provided. Similarly, only a first voltage pulse set 1002a1 and a second voltage pulse set 1002a2 delivered by the second subset of electrodes 315s2 are shown for voltage waveform 1002, although additional voltage pulse sets may be provided. For each of the voltage waveforms 1003 and 1004, only a single voltage pulse set is illustrated in FIG. 10, although additional voltage pulse sets may be provided.

Each voltage pulse set may be considered a portion of its respective second energy delivered by its respective subset of electrodes. For instance, according to some embodiments, first voltage pulse set 1001a1 may be considered a first portion of the respective second energy delivered by the first subset 315s1 of electrodes pursuant to block 506 in FIG. 5, and second of voltage pulse set 1001a2 may be considered a second portion of the respective second energy delivered by the first subset 315s1 of electrodes pursuant to block 506. Similarly, according to some embodiments, first voltage pulse set 1002a1 may be considered a first portion of the respective second energy delivered by the second subset 315s2 of electrodes pursuant to block 506, and second of voltage pulse set 1002a2 may be considered a second portion of the respective second energy delivered by the second subset 315s2 of electrodes pursuant to block 506.

In the example FIG. 10, each voltage pulse set is illustrated as a sequence of two biphasic voltage pulses. For instance, first voltage pulse set 1001a1 of voltage waveform 1001 is a sequence of two biphasic voltage pulses separated by an inter-pulse delay 1001b. In some embodiments, the inter-pulse delay is zero. In some embodiments, each voltage pulse set may contain different numbers of pulses than those illustrated in FIG. 10. In some embodiments, each voltage pulse set may be a pulse set of only a single voltage pulse, such as a single biphasic voltage pulse in some embodiments. In some embodiments, at least two of the voltage pulse sets contain, or are made up of, an equal number of pulses. In some embodiments, at least two of the voltage pulse sets contain, or are made up of, a different number of pulses.

In some embodiments, each respective second energy delivered according to block 506 in FIG. 5 is configured as a sequence of voltage pulse sets. For instance, in the example of FIG. 10, the sequence of voltage pulse sets 1001 delivered by the first subset 315s1 of electrodes includes a sequence of first voltage pulse set 1001a1 followed in the sequence by second voltage pulse set 1001a2, which may be followed by additional voltage pulse sets not shown in FIG. 10. The voltage pulse sets in the sequence are separated by an inter-pulse-set delay, according to some embodiments. For instance, in the example of FIG. 10, the first voltage pulse set 1001a1 is separated by the second voltage pulse set 1001a2 in the sequence of voltage pulse sets by an inter-pulse-set delay 1001c. In some embodiments, each of such inter-pulse-set delays is greater in duration than each of the inter-pulse delays between pulses within each voltage pulse set. For instance, in the example of FIG. 10, inter-pulse-set delay 1001c is greater than inter-pulse delay 1001b. Stated differently, in some embodiments, each respective second energy delivered according to block 506 in FIG. 5 is configured as a sequence of voltage pulse sets, such that an inter-pulse delay between pulses in each voltage pulse set in the sequence of voltage pulse sets is less than an inter-pulse-set delay between voltage pulse sets in the sequence of voltage pulse sets. In some embodiments, a duration of the inter-pulse-set delay may vary with the number of subsets of electrodes. For example, greater numbers of subsets of electrodes will, in some embodiments, increase the cycling time 1006 exemplified in FIG. 10, and as such, increase the inter-pulse-set delay.

In some embodiments, the separate subsets of electrodes include at least a first subset (e.g., 315s1) of electrodes and a second subset (e.g., 315s2) of electrodes. In some embodiments, block 506 may be associated with computer-executable instructions configured to cause the data processing device system (e.g., 110, 310) to cause, via the input-output device system (e.g., input-output device system 120, 320 or energy source device system 340) the first group of electrodes (e.g., 315G) to non-concurrently deliver, in the separate subsets of electrodes, the respective second energies to cause pulsed field ablation of bodily tissue including causing: (a) a delivery of a first portion (e.g., first voltage pulse set 1002a1 in FIG. 10, in some embodiments) of the respective second energy delivered by the second subset (e.g., 315s2) of electrodes to be delivered after a delivery of a first portion (e.g., first voltage pulse set 1001a1, in some embodiments) of the respective second energy delivered by the first subset (e.g., 315s1) of electrodes, and (b) a delivery of a second portion (e.g., second voltage pulse set 1001a2, in some embodiments) of the respective second energy delivered by the first subset (e.g., 315s1) of electrodes to be delivered after the delivery of the first portion (e.g., first voltage pulse set 1002a1 in FIG. 10, in some embodiments) of the respective second energy delivered by the second subset (e.g., 315s2) of electrodes.

In some embodiments, the first portion (e.g., first voltage pulse set 1001a1, in some embodiments) of the respective second energy delivered by the first subset (e.g., 315s1) of electrodes may be considered a first voltage pulse set, the first portion (e.g., first voltage pulse set 1002a1 in FIG. 10, in some embodiments) of the respective second energy delivered by the second subset (e.g., 315s2) of electrodes may be considered a second voltage pulse set, and the second portion (e.g., second voltage pulse set 1001a2, in some embodiments) of the respective second energy delivered by the first subset (e.g., 315s1) of electrodes may be considered a third voltage pulse set. In at least this context, in some embodiments, block 506 may be associated with computer-executable instructions configured to cause the data processing device system (e.g., 110, 310) to cause, via the input-output device system (e.g., input-output device system 120, 320 or energy source device system 340) the first group of electrodes (e.g., 315G) to non-concurrently deliver, in the separate subsets of electrodes, the respective second energies to cause pulsed field ablation of bodily tissue including causing: (a) a delivery of the second voltage pulse set (e.g., voltage pulse set 1002a1 in FIG. 10) of the respective second energy delivered by the second subset (e.g., 315s2) of electrodes to be delivered after a delivery of the first voltage pulse set (e.g., voltage pulse set 1001a1) of the respective second energy delivered by the first subset (e.g., 315s1) of electrodes, and (b) a delivery of the third voltage pulse set (e.g., voltage pulse set 1001a2) of the respective second energy delivered by the first subset (e.g., 315s1) of electrodes to be delivered after the delivery of the second voltage pulse set (e.g., voltage pulse set 1002a1) of the respective second energy delivered by the second subset (e.g., 315s2) of electrodes. Although the example of FIG. 10 shows voltage pulse sets (e.g., 1001a1, 1002a1, 1001a2, etc.) as each comprising two biphasic pulses, each of one or more of the voltage pulse sets may be a pulse set of more than two biphasic pulses or may be a pulse set of only a single voltage pulse, such as a single biphasic pulse, according to various embodiments. In this regard, for example, (i) the first voltage pulse set (e.g., voltage pulse set 1001a1), (ii) the second voltage pulse set (e.g., voltage pulse set 1002a1), (iii) the third voltage pulse set (e.g., voltage pulse set 1001a2), each of (i) and (ii), each of (i) and (iii), each of (ii) and (iii), or each of (i), (ii), and (iii) may, according to some embodiments, be a pulse set of only a single voltage pulse, such as a single biphasic voltage pulse.

In some embodiments, the causing the first group of electrodes to non-concurrently deliver, in the separate subsets of electrodes, the respective second energies to cause pulsed field ablation of bodily tissue per block 506 includes causing the separate subsets of electrodes to non-concurrently deliver the respective second energies at least by causing cycling or interleaving among the voltage pulse sets of the sequences of voltage pulse sets of the respective second energies. For instance, at least in the example of FIG. 10, the sequences of voltage pulse sets 1001-1004 are cycled or interleaved as an example implementation of the delivering of the respective second energies per block 506, in some embodiments. Although the example of FIG. 10 shows each of the sequences of voltage pulse sets 1001-1004 as including pairs of biphasic pulses as the pulse sets (see, e.g., at least pulse set 1001a1), some embodiments may have each of at least one voltage pulse set of the voltage pulse sets of the sequences of voltage pulse sets of the respective second energies as a pulse set of only a single voltage pulse (such as a single biphasic voltage pulse) or as a pulse set of more than two biphasic voltage pulses, according to various embodiments.

Although the example of FIG. 10 shows cycling or interleaving of voltage pulse sets 1001-1004 as an example implementation of the delivering of the respective second energies per block 506 among the respective subsets of electrodes (e.g., 315s1, 315s2, 315s3, 315s4), some embodiments may include delivering the respective second energy to each respective subset of electrodes per block 506 in FIG. 5 in sequence, such that, e.g., the first subset 315s1 delivers its entire second energy, then the second subset 315s2 delivers its entire second energy, then the third subset 315s3 delivers its entire second energy, and then the fourth subset 315s4 delivers its entire second energy.

As discussed above, in some embodiments, the first energy attempted to be delivered per block 502 in FIG. 5 may be predefined or predetermined (e.g., as recorded in memory device system 130, 330) to deliver a set number of voltage pulses. For instance, in the case of PFA, the first energy may be predefined or predetermined to have a set number of voltage pulses to be delivered over a predefined or predetermined period of time. In some of these and other embodiments, the respective second energies delivered according to block 506 in FIG. 5 may be configured to be the undelivered amount (e.g., number of voltage pulses) of the first energy that was unable to be delivered per block 502 due to the occurrence of the fault condition (identified per block 504). In some embodiments, the respective second energies may be configured to be within 10% of the portion of the first energy (e.g., number of voltage pulses) that was not delivered due at least to the identified fault condition. Such configurations may help ensure that the target tissue receives approximately the intended number of PFA voltage pulses in spite of the occurrence of the fault condition.

Also as discussed above, at least in some contexts or use cases, it may be advantageous to transmit the respective second energies non-concurrently to the respective separate subsets of electrodes in order to help isolate the electrode or electrodes in the first group of electrodes (e.g., 315G) that is or are involved in the fault condition, while contemporaneously allowing the medical procedure to safely proceed by delivering tissue-ablative energy to electrodes in the first group of electrodes that are not involved in the fault condition, thereby reducing medical procedure duration as compared to a case where the fault condition merely stops the medical procedure in its entirety.

In this regard, method 500 in FIG. 5 may include block 507 associated with computer-executable instructions (e.g., second identification instructions provided by a program) configured to cause the data processing device system (e.g., 110, 310) to identify that the fault condition has occurred in a particular subset of the separate subsets of electrodes at least in response to at least the particular subset of the separate subsets of electrodes attempting to deliver the respective second energy. Block 507 in FIG. 5 is shown in broken line to accentuate that it is an optional part of method 500 in some embodiments. Although not shown in FIG. 5, block 507 may be preceded with a block the same as or similar to block 503, where a signal set is received by the data processing device system, and such signal set is analyzed by the data processing device system to identify that the fault condition has occurred per block 507, in some embodiments. In some embodiments, block 507 may be executed to isolate the location of the cause of the fault condition originally detected per block 504, for example, when the particular subset of electrodes associated with block 507 may be identified as including one or more electrodes that may be the cause of the fault condition. When the fault condition is detected to occur in the particular subset of electrodes, transmission of the respective energy to such particular subset of electrodes may be terminated at least until the fault condition is removed or cleared.

In some embodiments, method 500 in FIG. 5 may include block 508 associated with computer-executable instructions (e.g., third energy delivery instructions provided by a program) configured to cause the data processing device system (e.g., 110, 310) to cause, via the input-output device system and in response to identifying that the fault condition has occurred in the particular subset of the separate subsets of electrodes per block 507, another electrode set to deliver third energy. Block 508 in FIG. 5 is shown in broken line to accentuate that it is an optional part of method 500 in some embodiments. In some embodiments, the third energy is or includes at least the respective second energy, or a remaining portion (e.g., a remaining number of voltage pulses) thereof, that was unable to be delivered due to the occurrence of the fault condition in response to the attempted delivery of the respective second energy by the particular subset of the separate subsets of electrodes. In some embodiments, the another electrode set caused to deliver the third energy per block 508 is or includes a neighboring electrode to an electrode in the particular subset of electrodes that was identified (e.g., per block 507) to have caused the fault condition. In some embodiments, the another electrode set caused to deliver the third energy per block 508 includes at least one electrode in another one of the separate subsets of electrodes (e.g., see block 506) other than the particular subset of electrodes that was identified (e.g., per block 507) to have caused the fault condition. In some embodiments, the another electrode set caused to deliver the third energy per block 508 is or includes a subset of electrodes that is not part of (e.g., is other than) the separate subsets of electrodes (e.g., see block 506). In some embodiments, the another electrode set may be identified to a user via a user-interface of the input-output device system for confirmation by the user before delivering the third energy.

In this regard, the activation of the another electrode set per block 508 to deliver the third energy may be a remedial action, in some embodiments, to make a best backup or alternate attempt to ablate the target tissue that was unable to be ablated by at least part of the particular subset of electrodes that was identified to have caused the fault condition (e.g., per block 507). In this regard, the another electrode set per block 508 may, in some embodiments, be a next best electrode set besides the particular subset of electrodes capable of ablating the target tissue that was unable to be ablated by at least part of the particular subset of electrodes that was identified to have caused the fault condition (e.g., per block 507).

Subsets or combinations of various embodiments described above provide further embodiments. These and other changes can be made to the invention in light of the above detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the claims.

What is claimed is:

1. A medical system comprising:
a data processing device system;
an input-output device system communicatively connected to the data processing device system, the input-output device system communicatively connectable to a plurality of electrodes supported by a structure of a catheter; and
a memory device system communicatively connected to the data processing device system and storing a program executable by the data processing device system, the data processing device system configured by the program at least to:
cause, via the input-output device system, a first group of electrodes from the plurality of electrodes to concurrently and collectively deliver as a group first energy configured to cause pulsed field ablation of bodily tissue;
identify, at least in response to the first group of electrodes concurrently and collectively attempting to deliver as a group the first energy to cause pulsed field ablation of bodily tissue, that a fault condition has occurred; and
cause, via the input-output device system and in response to identifying that the fault condition has occurred, the first group of electrodes to non-concurrently deliver, in separate subsets of electrodes, respective second energies to cause pulsed field ablation of bodily tissue, the separate subsets of electrodes comprising electrodes that collectively make up the first group of electrodes.

2. The medical system of claim 1, wherein each of the separate subsets of electrodes is a subset of two or more electrodes.

3. The medical system of claim 1, wherein each of the separate subsets of electrodes is a subset of an even number of electrodes.

4. The medical system of claim 1, wherein each of the separate subsets of electrodes comprises at least one electrode that is other than another electrode in another of the separate subsets of electrodes.

5. The medical system of claim 1, wherein each of the separate subsets of electrodes does not comprise any electrode that is also included in any of the other separate subsets of electrodes.

6. The medical system of claim 1, wherein the first group of electrodes comprises four or more electrodes.

7. The medical system of claim 1, wherein the data processing device system is configured by the program at least to:
receive, via the input-output device system, a signal set; and
execute the identifying that the fault condition has occurred based at least on an analysis of the received signal set.

8. The medical system of claim 7,
wherein the data processing device system is configured by the program at least to receive, via the input-output device system, the signal set from an electrode set,
wherein the data processing device system is communicatively connected to the plurality of electrodes via the input-output device system, and wherein the electrode set is from the plurality of electrodes, and
wherein the electrode set is from the first group of electrodes.

9. The medical system of claim 1, wherein the fault condition is an overcurrent condition or a low impedance condition.

10. The medical system of claim 1, wherein the fault condition is a low current condition or a high impedance condition.

11. The medical system of claim 1,
wherein the input-output device system is communicatively connected to an energy source device system, the energy source device system comprising a single power delivery driver configured to transmit energy to the first group of electrodes, and wherein the causing the first group of electrodes from the plurality of electrodes to concurrently and collectively deliver as a group the first energy configured to cause pulsed field ablation of bodily tissue includes causing the single power delivery driver to transmit the first energy to the first group of electrodes, and
wherein the causing the first group of electrodes to non-concurrently deliver, in the separate subsets of electrodes, the respective second energies to cause pulsed field ablation of bodily tissue includes causing the single power delivery driver to transmit the respective second energies to the separate subsets of electrodes.

12. The medical system of claim 1, wherein the separate subsets of electrodes comprise at least a first subset of electrodes and a second subset of electrodes, and wherein the causing the first group of electrodes to non-concurrently deliver, in the separate subsets of electrodes, the respective second energies to cause pulsed field ablation of bodily tissue includes causing: (a) a delivery of a first portion of the respective second energy delivered by the second subset of electrodes to be delivered after a delivery of a first portion of the respective second energy delivered by the first subset of electrodes, and (b) a delivery of a second portion of the respective second energy delivered by the first subset of electrodes to be delivered after the delivery of the first portion of the respective second energy delivered by the second subset of electrodes.

13. The medical system of claim 12, wherein the first portion of the respective second energy delivered by the first subset of electrodes is a first voltage pulse set, the first portion of the respective second energy delivered by the second subset of electrodes is a second voltage pulse set, and the second portion of the respective second energy delivered by the first subset of electrodes is a third voltage pulse set.

14. The medical system of claim 13, wherein (i) the first voltage pulse set, (ii) the second voltage pulse set, (iii) the third voltage pulse set, each of (i) and (ii), each of (i) and (iii), each of (ii) and (iii), or each of (i), (ii), and (iii) is a pulse set of only a single voltage pulse.

15. The medical system of claim 14, wherein the single voltage pulse is a single biphasic voltage pulse.

16. The medical system of claim 1, wherein the respective second energies are configured to be within 10% of a portion of the first energy that was not delivered due at least to the identified fault condition.

17. The medical system of claim 1,
wherein each respective second energy is configured as a sequence of voltage pulse sets, an inter-pulse delay between pulses in each voltage pulse set in the sequence of voltage pulse sets less than an inter-pulse-set delay between voltage pulse sets in the sequence of voltage pulse sets, and
wherein the causing the first group of electrodes to non-concurrently deliver, in the separate subsets of electrodes, the respective second energies to cause pulsed field ablation of bodily tissue includes causing the separate subsets of electrodes to non-concurrently deliver the respective second energies at least by causing cycling among the voltage pulse sets of the sequences of voltage pulse sets of the respective second energies.

18. The medical system of claim 1,
wherein each respective second energy is configured as a sequence of voltage pulse sets, an inter-pulse delay between pulses in each voltage pulse set in the sequence of voltage pulse sets less than an inter-pulse-set delay between voltage pulse sets in the sequence of voltage pulse sets, and
wherein the causing the first group of electrodes to non-concurrently deliver, in the separate subsets of electrodes, the respective second energies to cause pulsed field ablation of bodily tissue includes causing the separate subsets of electrodes to non-concurrently deliver the respective second energies at least by interleaving the voltage pulse sets of the sequences of voltage pulse sets of the respective second energies.

19. The medical system of claim 1,
wherein each respective second energy is configured as a sequence of voltage pulse sets, an inter-pulse delay between pulses in each voltage pulse set in the sequence of voltage pulse sets less than an inter-pulse-set delay between voltage pulse sets in the sequence of voltage pulse sets, and
wherein each of at least one voltage pulse set of the voltage pulse sets of the sequences of voltage pulse sets of the respective second energies is a pulse set of only a single voltage pulse.

20. The medical system of claim 19, wherein the single voltage pulse is a single biphasic voltage pulse.

21. The medical system of claim 1, wherein the data processing device system is configured by the program at least to identify that the fault condition has occurred in a particular subset of the separate subsets of electrodes at least in response to at least the particular subset of the separate subsets of electrodes attempting to deliver the respective second energy.
